SEQUENCE LISTING (30 sequences)

SEQ ID NO: 1 - DNA, 23 bp, synthetic construct
  misc_feature 1..23: note = CpG oligonucleotide
  source 1..23: mol_type = other DNA
tcgtcgacgatcggcgcgcgccg

SEQ ID NO: 2 - DNA, 23 bp, synthetic construct
  misc_feature 1..23: note = CpG oligonucleotide
  source 1..23: mol_type = other DNA
tcgacgtcgatcggcgcgcgccg

SEQ ID NO: 3 - DNA, 24 bp, synthetic construct
  misc_feature 1..24: note = CpG oligonucleotide
  source 1..24: mol_type = other DNA
tcgacgtcgatcggcgcgcgccgt

SEQ ID NO: 4 - DNA, 23 bp, synthetic construct
  misc_feature 1..23: note = CpG oligonucleotide
  misc_feature 1: note = 5'-Iodo-2'-deoxyuridine
  source 1..23: mol_type = other DNA
ncgacgtcgatcggcgcgcgccg

SEQ ID NO: 5 - DNA, 24 bp, synthetic construct
  misc_feature 1..24: note = CpG oligonucleotide
  misc_feature 1: note = 5'-Iodo-2'-deoxyuridine
  source 1..24: mol_type = other DNA
ncgacgtcgatcggcgcgcgccgt

SEQ ID NO: 6 - DNA, 24 bp, synthetic construct
  misc_feature 1..24: note = CpG oligonucleotide
  misc_feature 1: note = 5'-Iodo-2'-deoxyuridine
  source 1..24: mol_type = other DNA
ncgacgtcgatcggcgcgcgccgt

SEQ ID NO: 7 - DNA, 23 bp, synthetic construct
  misc_feature 1..23: note = CpG oligonucleotide
  misc_feature 1: note = 5'-Ethyl-2'-deoxyuridine
  source 1..23: mol_type = other DNA
ncgacgtcgatcggcgcgcgccg

SEQ ID NO: 8 - DNA, 24 bp, synthetic construct
  misc_feature 1..24: note = CpG oligonucleotide
  misc_feature 1: note = 5'-Iodo-2'-deoxyuridine
  source 1..24: mol_type = other DNA
ncgtcgacgatcggcggccgccgt

SEQ ID NO: 9 - DNA, 24 bp, synthetic construct
  misc_feature 1..24: note = CpG oligonucleotide
  misc_feature 1: note = 5'-Iodo-2'-deoxyuridine
  source 1..24: mol_type = other DNA
ncgtcgacgatcggcggccgccgt

SEQ ID NO: 10 - DNA, 23 bp, synthetic construct
  misc_feature 1..23: note = CpG oligonucleotide
  source 1..23: mol_type = other DNA
tcgtcgacgatcggcgcgcgccg

SEQ ID NO: 11 - DNA, 20 bp, synthetic construct
  misc_feature 1..20: note = RNA
  source 1..20: mol_type = other DNA
ttgttgttgttgttgttgtt

SEQ ID NO: 12 - DNA, 20 bp, synthetic construct
  misc_feature 1..20: note = RNA
  source 1..20: mol_type = other DNA
ttattattattattattatt

SEQ ID NO: 13 - DNA, 19 bp, synthetic construct
  misc_feature 1..19: note = RNA
  source 1..19: mol_type = other DNA
aaacgctcagccaaagcag

SEQ ID NO: 14 - DNA, 21 bp, synthetic construct
  misc_feature 1..21: note = DNA/RNA
  misc_feature 11..17: note = ribonucleotides
  source 1..21: mol_type = other DNA
tcgtcgttttgttgtgttttt

SEQ ID NO: 15 - DNA, 10867 bp, synthetic construct
  misc_feature 1..10867: note = Fusion nucleotide: Foot and Mouth Disease Virus (FMDV) and Bovine Rhinovirus Type 2 (BRV2)
  source 1..10867: mol_type = other DNA
ggggccggccaatccagtccggcgaccggctcgcagaaccaatctggcaacactggcagcataattaacaactactacatgcagcaataccagaactccatggacacacagttgggagacaatgccatcagtggaggctccaacgagggctccacggacacaacttcaacacacacaaccaacactcaaaacaatgactggttctcgaagctcgccagttcagcttttaccggtctgttcggtgcactgctcgccgacaagaagacagaggaaacgacacttcttgaggaccgcatcctcaccacccgcaacgggcacaccacctcgacgacccaatcgagtgtgggtgtcacacacgggtactccacagaggaggaccacgttgctgggcccaacacatcgggcctggagacgcgagtggtgcaggcagagagattctacaaaaagtacttgtttgactggacaacggacaaggcatttggacacctggaaaagctggagctcccgtccgaccaccacggtgtctttggacacttggtggactcgtacgcctatatgagaaatggctgggatgttgaggtgtccgctgttggcaaccagttcaacggcgggtgcctcctggtggccatggtacctgaatggaaggaatttgacacacgggagaaataccaactcacccttttcccgcaccagtttattagccccagaactaacatgactgcccacatcacggtcccctaccttggtgtgaacaggtatgatcagtacaagaagcataagccctggacattggttgtcatggtcgtgtcgccacttacggtcaacaacactagtgcggcacaaatcaaggtctacgccaacatagctccgacctatgttcacgtggccggtgaactcccctcgaaagaggggattttcccggttgcatgtgcggacggttacggaggattggtgacgacagacccgaagacagctgaccctgcttatggcaaggtgtacaacccgcctaggactaactaccctgggcgcttcaccaacctgttggacgtggccgaagcgtgtcccactttcctctgctttgacgacgggaaaccgtacgtcaccacgcggacggatgacacccgacttttggccaagtttgacctttcccttgccgcaaaacatatgtccaacacatacctgtcagggattgctcagtactacacacagtactctggcaccatcaatttgcatttcatgttcacaggttccactgattcaaaggcccgatacatggtggcctacatcccacctggggtggagacaccaccggacacacctgaaagggctgcccactgcattcacgctgaatgggacactggactaaactccaaattcactttctcaatcccgtacgtatccgccgcggattacgcgtacacagcgtctgacacggcagaaacaatcaacgtacagggatgggtctgcatctaccaaattacacacgggaaggctgaaaatgacaccttggtcgtgtcggttagcgccggcaaagactttgagttgcgcctcccgattgacccccgccagcagaccaccgctaccggggaatcagcagacccggtcaccaccaccgtggagaactacggcggtgagacacaaatccagagacgtcaccacacggacattggtttcatcatggacagatttgtgaagatccaaagcttgagcccaacacatgtcattgacctcatgcagactcaccaacacggtctggtgggtgccttgctgcgtgcagccacgtactacttttctgacctggaaattgttgtacggcacgaaggcaatctgacctgggtgcccaacggcgcccctgaatcagccctgttgaacaccagcaaccccactgcctacaacaaggcaccattcacgagactcgctctcccctacactgcgccgcaccgtgtgctggcaacagtgtacaacgggacgagtaagtatgctgtgggtggttcaggcagaagaggcgacatggggtctctcgcggcgcgagtcgtgaaacagcttcctgcttcatttaactacggtgcaatcaaggccgacgccatccacgaacttctcgtgcgcatgaaacgggccgagctctactgccccagaccgctgttggcaatagaggtgtcttcgcaagacaggcacaagcaaaagatcattgcaccagcaaagcagcttctgaattttgacctgcttaagctagccggagacgttgagtccaaccctgggcccttcttcttctccgacgttaggtcaaacttttccaagctggtagacacaatcaaccagatgcaggaagacatgtccacaaagcacggacctgactttaaccggttggtgtccgcttttgaggagttggccactggagtgaaagccatcaggaccggtcttgacgaggccaagccctggtacaagcttatcaagctcctgagccgcctgtcgtgcatggccgctgtggcagcacggtcaaaggacccagtccttgtggccatcatgctggctgacaccggtctcgagattctggacagcaccttcgtcgtgaagaagatctccgactcgctctccagtctcttccacgtgccggcccccgtcttcagtttcggagccccgattctgttagccgggttggtcaaggtcgcctcgagtttcttccggtccacgcccgaagaccttgagagagcagagaaacagctcaaagcacgtgacatcaacgacattttcgccattctcaagaacggcgagtggctggtcaaattgatccttgccatccgcgactggatcaaggcatggatagcctcagaagaaaagtttgtcaccacgacagacttggtacctagcatccttgaaaaacagcaggacctcaacgacccaagcaagtacaaggaagccaaggagtggctcgacaacgcgcgccaagcgtgtttgaagagcgggaacgtccacattgccaacctgtgcaaagtggtcgccccggcacccagcaggtcgagacccgagcccgtggtcgtttgcctccgtggcaagtccggtcagggcaagagtttccttgcaaacgtgctcgcacaagcaatctctacccatttcactggcaggaccgattcagtttggtactgcccgcctgaccctgaccacttcgacggttacaaccaacagactgtcgttgtgatggacgatttgggccagaaccccgacggcaaagacttcaagtacttcgcccaaatggtttcaacaacggggttcatcccgcccatggcatcgcttgaggataaaggcaaacccttcaacagtaaggtcatcatagcaaccaccaacctgtactcgggcttcaccccgaggactatggtgtgccctgatgccctgaaccggaggtttcactttgacatcgacgtgagcgccaaggacgggtacaaaattaacaacaaattggacatcatcaaagcacttgaagatactcacaccaacccagtggcaatgtttcagtacgactgtgcccttctcaacggcatggctgttgaaatgaagagaatgcaacaagatatgttcaagcctcaaccaccccttcagaacgtgtaccaactggttcaagaggtgattgagcgggtggagctccacgagaaggtgtcgagccacccgattttcaaacagatctcaattccttcccaaaaatccgtgttgtacttcctcattgagaaaggacagcacgaggcagcaattgaattctttgagggcatggtgcacgactccatcaaggaggagctccggccgctcatccaacaaacctcatttgtgaaacgcgcttttaagcgcctgaaggaaaactttgagattgttgccctatgtctgaccctcctggccaacatagtgatcatgatccgcgaaactcgcaagagacagaagatggtggacgatgcagtgagtgagtacattgagagagcaaacatcaccaccgacgacaagactcttgatgaggcggaaaagaaccctctggaaaccagcggtgccagcaccgtcggcttcagagagagacctctcccaggccaaaaggcgcgtaatgacgagaactccgagcccgcccagcctgctgaagagcaaccacaagctgaaggaccctacgctggcccgatggagagaccagttaaagttaaagtgaaagcaaaagccccggtcgttaaggaaggaccttacgagggaccggtgaagaagcctgttgctttgaaagtgaaagctaagaacttgatcgtcactgagagtggtgccccaccgaccgacttgcaaaagttggtcatgggcaacaccaagcccgttgagctcatccttgacgggaagacggtagccatttgctgtgctactggagttttcggcactgcttacctcgtgcctcgtcatcttttcgcagaaaagtacgacaagatcatgttggacggcagagccatgacagatagtgactacagagtgtttgagtttgagattaaagtaaaaggacaggacatgctctcagacgctgcgctcaggggccggccaatccagtccggcgaccggctcgcagaaccaatctggcaacactggcagcataattaacaactactacatgcagcaataccagaactccatggacacacagttgggagacaatgccatcagtggaggctccaacgagggctccacggacacaacttcaacacacacaaccaacactcaaaacaatgactggttctcgaagctcgccagttcagcttttaccggtctgttcggtgcactgctcgccgacaagaagacagaggaaacgacacttcttgaggaccgcatcctcaccacccgcaacgggcacaccacctcgacgacccaatcgagtgtgggtgtcacacacgggtactccacagaggaggaccacgttgctgggcccaacacatcgggcctggagacgcgagtggtgcaggcagagagattctacaaaaagtacttgtttgactggacaacggacaaggcatttggacacctggaaaagctggagctcccgtccgaccaccacggtgtctttggacacttggtggactcgtacgcctatatgagaaatggctgggatgttgaggtgtccgctgttggcaaccagttcaacggcgggtgcctcctggtggccatggtacctgaatggaaggaatttgacacacgggagaaataccaactcacccttttcccgcaccagtttattagccccagaactaacatgactgcccacatcacggtcccctaccttggtgtgaacaggtatgatcagtacaagaagcataagccctggacattggttgtcatggtcgtgtcgccacttacggtcaacaacactagtgcggcacaaatcaaggtctacgccaacatagctccgacctatgttcacgtggccggtgaactcccctcgaaagaggggattttcccggttgcatgtgcggacggttacggaggattggtgacgacagacccgaagacagctgaccctgcttatggcaaggtgtacaacccgcctaggactaactaccctgggcgcttcaccaacctgttggacgtggccgaagcgtgtcccactttcctctgctttgacgacgggaaaccgtacgtcaccacgcggacggatgacacccgacttttggccaagtttgacctttcccttgccgcaaaacatatgtccaacacatacctgtcagggattgctcagtactacacacagtactctggcaccatcaatttgcatttcatgttcacaggttccactgattcaaaggcccgatacatggtggcctacatcccacctggggtggagacaccaccggacacacctgaaagggctgcccactgcattcacgctgaatgggacactggactaaactccaaattcactttctcaatcccgtacgtatccgccgcggattacgcgtacacagcgtctgacacggcagaaacaatcaacgtacagggatgggtctgcatctaccaaattacacacgggaaggctgaaaatgacaccttggtcgtgtcggttagcgccggcaaagactttgagttgcgcctcccgattgacccccgccagcagaccaccgctaccggggaatcagcagacccggtcaccaccaccgtggagaactacggcggtgagacacaaatccagagacgtcaccacacggacattggtttcatcatggacagatttgtgaagatccaaagcttgagcccaacacatgtcattgacctcatgcagactcaccaacacggtctggtgggtgccttgctgcgtgcagccacgtactacttttctgacctggaaattgttgtacggcacgaaggcaatctgacctgggtgcccaacggcgcccctgaatcagccctgttgaacaccagcaaccccactgcctacaacaaggcaccattcacgagactcgctctcccctacactgcgccgcaccgtgtgctggcaacagtgtacaacgggacgagtaagtatgctgtgggtggttcaggcagaagaggcgacatggggtctctcgcggcgcgagtcgtgaaacagcttcctgcttcatttaactacggtgcaatcaaggccgacgccatccacgaacttctcgtgcgcatgaaacgggccgagctctactgccccagaccgctgttggcaatagaggtgtcttcgcaagacaggcacaagcaaaagatcattgcaccagcaaagcagcttctgaattttgacctgcttaagctagccggagacgttgagtccaaccctgggcccttcttcttctccgacgttaggtcaaacttttccaagctggtagacacaatcaaccagatgcaggaagacatgtccacaaagcacggacctgactttaaccggttggtgtccgcttttgaggagttggccactggagtgaaagccatcaggaccggtcttgacgaggccaagccctggtacaagcttatcaagctcctgagccgcctgtcgtgcatggccgctgtggcagcacggtcaaaggacccagtccttgtggccatcatgctggctgacaccggtctcgagattctggacagcaccttcgtcgtgaagaagatctccgactcgctctccagtctcttccacgtgccggcccccgtcttcagtttcggagccccgattctgttagccgggttggtcaaggtcgcctcgagtttcttccggtccacgcccgaagaccttgagagagcagagaaacagctcaaagcacgtgacatcaacgacattttcgccattctcaagaacggcgagtggctggtcaaattgatccttgccatccgcgactggatcaaggcatggatagcctcagaagaaaagtttgtcaccacgacagacttggtacctagcatccttgaaaaacagcaggacctcaacgacccaagcaagtacaaggaagccaaggagtggctcgacaacgcgcgccaagcgtgtttgaagagcgggaacgtccacattgccaacctgtgcaaagtggtcgccccggcacccagcaggtcgagacccgagcccgtggtcgtttgcctccgtggcaagtccggtcagggcaagagtttccttgcaaacgtgctcgcacaagcaatctctacccatttcactggcaggaccgattcagtttggtactgcccgcctgaccctgaccacttcgacggttacaaccaacagactgtcgttgtgatggacgatttgggccagaaccccgacggcaaagacttcaagtacttcgcccaaatggtttcaacaacggggttcatcccgcccatggcatcgcttgaggataaaggcaaacccttcaacagtaaggtcatcatagcaaccaccaacctgtactcgggcttcaccccgaggactatggtgtgccctgatgccctgaaccggaggtttcactttgacatcgacgtgagcgccaaggacgggtacaaaattaacaacaaattggacatcatcaaagcacttgaagatactcacaccaacccagtggcaatgtttcagtacgactgtgcccttctcaacggcatggctgttgaaatgaagagaatgcaacaagatatgttcaagcctcaaccaccccttcagaacgtgtaccaactggttcaagaggtgattgagcgggtggagctccacgagaaggtgtcgagccacccgattttcaaacagatctcaattccttcccaaaaatccgtgttgtacttcctcattgagaaaggacagcacgaggcagcaattgaattctttgagggcatggtgcacgactccatcaaggaggagctccggccgctcatccaacaaacctcatttgtgaaacgcgcttttaagcgcctgaaggaaaactttgagattgttgccctatgtctgaccctcctggccaacatagtgatcatgatccgcgaaactcgcaagagacagaagatggtggacgatgcagtgagtgagtacattgagagagcaaacatcaccaccgacgacaagactcttgatgaggcggaaaagaaccctctggaaaccagcggtgccagcaccgtcggcttcagagagagacctctcccaggccaaaaggcgcgtaatgacgagaactccgagcccgcccagcctgctgaagagcaaccacaagctgaaggaccctacgctggcccgatggagagaccagttaaagttaaagtgaaagcaaaagccccggtcgttaaggaaggaccttacgagggaccggtgaagaagcctgttgctttgaaagtgaaagctaagaacttgatcgtcactgagagtggtgccccaccgaccgacttgcaaaagttggtcatgggcaacaccaagcccgttgagctcatccttgacgggaagacggtagccatttgctgtgctactggagttttcggcactgcttacctcgtgcctcgtcatcttttcgcagaaaagtacgacaagatcatgttggacggcagagccatgacagatagtgactacagagtgtttgagtttgagattaaagtaaaaggacaggacatgctctcagacgctgcgctcatggtgctccaccgtgggaatcgcgtgagagacatcacgaaacactttcgtgacacagcaagaatgaagaaaggcacccccgtcgttggtgtgatcaacaacgccgatgtcgggagactgattttctctggtgaagcccttacctacaaggacattgtagtgtgcatggatggagacaccatgcctgggctctttgcctacaaagccgcaaccaaggctggttattgcggaggagccgtcctcgctaaggacggggctgacacgttcatcgttggcacccactccgctggaggcaatggcgttggatactgctcttgcgtttccaggtccatgcttctcaagatgaaggcacacgttgaccccgaaccacaccacgaggggttgattgttgacaccagagatgtggaagagcgcgttcacgtgatgcgcaaaaccaagcttgcacccaccgttgcgtacggtgtgttccgtcctgagttcgggcctgccgccttgtccaacaaggacccgcgcctgaacgacggtgttgtcctcgacgaagtcatcttctccaaacacaagggagacacaaagatgtctgaggaagacaaagcgctgttccgccgctgtgctgctgactacgcgtcacgcctgcacagcgtgttgggtacggcaaatgccccactgagcatctacgaggcaattaaaggcgttgatggactcgacgcaatggaaccagacaccgcacccggcctcccctgggcactccaggggaagcgccgtggcgcgctcatcgacttcgagaacggcactgttggacccgaagttgaggctgccttgaagctcatggagaaaagagaatacaagtttgcttgccaaaccttcctgaaggacgagattcgcccgatggagaaagtacgtgccggtaagactcgcattgtcgacgtcctacctgttgaacacatcctctacaccaggatgatgattggcagattttgtgcacaaatgcactcaaacaacggaccccaaattggctcggcggtcggttgtaaccctgatgttgattggcaaagatttggcacacacttcgcccaatacagaaacgtgtgggatgtggactattcggccttcgatgctaaccactgcagtgacgccatgaacatcatgtttgaggaagtgtttcgcacagaattcgggttccacccaaacgctgagtggatcctgaagactctcgtgaacacggaacacgcctatgagaacaaacgcatcactgttgaaggcgggatgccatctggttgttccgcaacaagcatcatcaacacaattttgaacaacatctacgtgctctacgctttgcgtagacactatgagggagttgagctggacacttacaccatgatctcttacggagacgatatcgtggtggcaagtgattacgatttggactttgaggctctcaagccccacttcaaatcccttggtcaaaccatcactccagctgacaaaagcgacaaaggttttgttcttggtcactccattactgatgtcactttcctcaaaagacacttccacatggattatggaactgggttttacaaacctgtgatggcctcaaagacccttgaggctatcctctcctttgcacgccgtgggaccatacaggagaagttgatctccgtggcaggactcgctgttcactctggaccagacgagtaccggcgtctcttcgagccctttcaaggcctcttcgagattccaagctacagatcactttacctgcgttgggtgaacgccgtgtgcggcgacgca

SEQ ID NO: 16 - protein, 2109 aa, synthetic construct
  REGION 1..2109: note = Fusion protein: Foot and Mouth Disease Virus (FMDV) and Bovine Rhinovirus Type 2 (BRV2)
GAGQSSPATGSQNQSGNTGSIINNYYMQQYQNSMDTQLGDNAISGGSNEGSTDTTSTHTTNTQNNDWFSKLASSAFTGLFGALLADKKTEETTLLEDRILTTRNGHTTSTTQSSVGVTHGYSTEEDHVAGPNTSGLETRVVQAERFYKKYLFDWTTDKAFGHLEKLELPSDHHGVFGHLVDSYAYMRNGWDVEVSAVGNQFNGGCLLVAMVPEWKEFDTREKYQLTLFPHQFISPRTNMTAHITVPYLGVNRYDQYKKHKPWTLVVMVVSPLTVNNTSAAQIKVYANIAPTYVHVAGELPSKEGIFPVACADGYGGLVTTDPKTADPAYGKVYNPPRTNYPGRFTNLLDVAEACPTFLCFDDGKPYVTTRTDDTRLLAKFDLSLAAKHMSNTYLSGIAQYYTQYSGTINLHFMFTGSTDSKARYMVAYIPPGVETPPDTPERAAHCIHAEWDTGLNSKFTFSIPYVSAADYAYTASDTAETINVQGWVCIYQITHGKAENDTLVVSVSAGKDFELRLPIDPRQQTTATGESADPVTTTVENYGGETQIQRRHHTDIGFIMDRFVKIQSLSPTHVIDLMQTHQHGLVGALLRAATYYFSDLEIVVRHEGNLTWVPNGAPESALLNTSNPTAYNKAPFTRLALPYTAPHRVLATVYNGTSKYAVGGSGRRGDMGSLAARVVKQLPASFNYGAIKADAIHELLVRMKRAELYCPRPLLAIEVSSQDRHKQKIIAPAKQLLNFDLLKLAGDVESNPGPFFFSDVRSNFSKLVDTINQMQEDMSTKHGPDFNRLVSAFEELATGVKAIRTGLDEAKPWYKLIKLLSRLSCMAAVAARSKDPVLVAIMLADTGLEILDSTFVVKKISDSLSSLFHVPAPVFSFGAPILLAGLVKVASSFFRSTPEDLERAEKQLKARDINDIFAILKNGEWLVKLILAIRDWIKAWIASEEKFVTTTDLVPSILEKQQDLNDPSKYKEAKEWLDNARQACLKSGNVHIANLCKVVAPAPSRSRPEPVVVCLRGKSGQGKSFLANVLAQAISTHFTGRTDSVWYCPPDPDHFDGYNQQTVVVMDDLGQNPDGKDFKYFAQMVSTTGFIPPMASLEDKGKPFNSKVIIATTNLYSGFTPRTMVCPDALNRRFHFDIDVSAKDGYKINNKLDIIKALEDTHTNPVAMFQYDCALLNGMAVEMKRMQQDMFKPQPPLQNVYQLVQEVIERVELHEKVSSHPIFKQISIPSQKSVLYFLIEKGQHEAAIEFFEGMVHDSIKEELRPLIQQTSFVKRAFKRLKENFEIVALCLTLLANIVIMIRETRKRQKMVDDAVSEYIERANITTDDKTLDEAEKNPLETSGASTVGFRERPLPGQKARNDENSEPAQPAEEQPQAEGPYAGPMERPVKVKVKAKAPVVKEGPYEGPVKKPVALKVKAKNLIVTESGAPPTDLQKLVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGNGVGYCSCVSRSMLLKMKAHVDPEPHHEGLIVDTRDVEERVHVMRKTKLAPTVAYGVFRPEFGPAALSNKDPRLNDGVVLDEVIFSKHKGDTKMSEEDKALFRRCAADYASRLHSVLGTANAPLSIYEAIKGVDGLDAMEPDTAPGLPWALQGKRRGALIDFENGTVGPEVEAALKLMEKREYKFACQTFLKDEIRPMEKVRAGKTRIVDVLPVEHILYTRMMIGRFCAQMHSNNGPQIGSAVGCNPDVDWQRFGTHFAQYRNVWDVDYSAFDANHCSDAMNIMFEEVFRTEFGFHPNAEWILKTLVNTEHAYENKRITVEGGMPSGCSATSIINTILNNIYVLYALRRHYEGVELDTYTMISYGDDIVVASDYDLDFEALKPHFKSLGQTITPADKSDKGFVLGHSITDVTFLKRHFHMDYGTGFYKPVMASKTLEAILSFARRGTIQEKLISVAGLAVHSGPDEYRRLFEPFQGLFEIPSYRSLYLRWVNAVCGDA

SEQ ID NO: 17 - DNA, 6327 bp, synthetic construct
  misc_feature 1..6327: note = Fusion nucleotide: Foot and Mouth Disease Virus (FMDV) and Bovine Rhinovirus Type 2 (BRV2)
  source 1..6327: mol_type = other DNA
ggggccggccaatccagtccggcgaccggctcgcagaaccaatctggcaacactggcagcataattaacaactactacatgcagcaataccagaactccatggacacacagttgggagacaatgccatcagtggaggctccaacgagggctccacggacacaacttcaacacacacaaccaacactcaaaacaatgactggttctcgaagctcgccagttcagcttttaccggtctgttcggtgcactgctcgccgacaagaagacagaggaaacgacacttcttgaggaccgcatcctcaccacccgcaacgggcacaccacctcgacgacccaatcgagtgtgggtgtcacacacgggtactccacagaggaggaccacgttgctgggcccaacacatcgggcctggagacgcgagtggtgcaggcagagagattctacaaaaagtacttgtttgactggacaacggacaaggcatttggacacctggaaaagctggagctcccgtccgaccaccacggtgtctttggacacttggtggactcgtacgcctatatgagaaatggctgggatgttgaggtgtccgctgttggcaaccagttcaacggcgggtgcctcctggtggccatggtacctgaatggaaggaatttgacacacgggagaaataccaactcacccttttcccgcaccagtttattagccccagaactaacatgactgcccacatcacggtcccctaccttggtgtgaacaggtatgatcagtacaagaagcataagccctggacattggttgtcatggtcgtgtcgccacttacggtcaacaacactagtgcggcacaaatcaaggtctacgccaacatagctccgacctatgttcacgtggccggtgaactcccctcgaaagaggggattttcccggttgcatgtgcggacggttacggaggattggtgacgacagacccgaagacagctgaccctgcttatggcaaggtgtacaacccgcctaggactaactaccctgggcgcttcaccaacctgttggacgtggccgaagcgtgtcccactttcctctgctttgacgacgggaaaccgtacgtcaccacgcggacggatgacacccgacttttggccaagtttgacctttcccttgccgcaaaacatatgtccaacacatacctgtcagggattgctcagtactacacacagtactctggcaccatcaatttgcatttcatgttcacaggttccactgattcaaaggcccgatacatggtggcctacatcccacctggggtggagacaccaccggacacacctgaaagggctgcccactgcattcacgctgaatgggacactggactaaactccaaattcactttctcaatcccgtacgtatccgccgcggattacgcgtacacagcgtctgacacggcagaaacaatcaacgtacagggatgggtctgcatctaccaaattacacacgggaaggctgaaaatgacaccttggtcgtgtcggttagcgccggcaaagactttgagttgcgcctcccgattgacccccgccagcagaccaccgctaccggggaatcagcagacccggtcaccaccaccgtggagaactacggcggtgagacacaaatccagagacgtcaccacacggacattggtttcatcatggacagatttgtgaagatccaaagcttgagcccaacacatgtcattgacctcatgcagactcaccaacacggtctggtgggtgccttgctgcgtgcagccacgtactacttttctgacctggaaattgttgtacggcacgaaggcaatctgacctgggtgcccaacggcgcccctgaatcagccctgttgaacaccagcaaccccactgcctacaacaaggcaccattcacgagactcgctctcccctacactgcgccgcaccgtgtgctggcaacagtgtacaacgggacgagtaagtatgctgtgggtggttcaggcagaagaggcgacatggggtctctcgcggcgcgagtcgtgaaacagcttcctgcttcatttaactacggtgcaatcaaggccgacgccatccacgaacttctcgtgcgcatgaaacgggccgagctctactgccccagaccgctgttggcaatagaggtgtcttcgcaagacaggcacaagcaaaagatcattgcaccagcaaagcagcttctgaattttgacctgcttaagctagccggagacgttgagtccaaccctgggcccttcttcttctccgacgttaggtcaaacttttccaagctggtagacacaatcaaccagatgcaggaagacatgtccacaaagcacggacctgactttaaccggttggtgtccgcttttgaggagttggccactggagtgaaagccatcaggaccggtcttgacgaggccaagccctggtacaagcttatcaagctcctgagccgcctgtcgtgcatggccgctgtggcagcacggtcaaaggacccagtccttgtggccatcatgctggctgacaccggtctcgagattctggacagcaccttcgtcgtgaagaagatctccgactcgctctccagtctcttccacgtgccggcccccgtcttcagtttcggagccccgattctgttagccgggttggtcaaggtcgcctcgagtttcttccggtccacgcccgaagaccttgagagagcagagaaacagctcaaagcacgtgacatcaacgacattttcgccattctcaagaacggcgagtggctggtcaaattgatccttgccatccgcgactggatcaaggcatggatagcctcagaagaaaagtttgtcaccacgacagacttggtacctagcatccttgaaaaacagcaggacctcaacgacccaagcaagtacaaggaagccaaggagtggctcgacaacgcgcgccaagcgtgtttgaagagcgggaacgtccacattgccaacctgtgcaaagtggtcgccccggcacccagcaggtcgagacccgagcccgtggtcgtttgcctccgtggcaagtccggtcagggcaagagtttccttgcaaacgtgctcgcacaagcaatctctacccatttcactggcaggaccgattcagtttggtactgcccgcctgaccctgaccacttcgacggttacaaccaacagactgtcgttgtgatggacgatttgggccagaaccccgacggcaaagacttcaagtacttcgcccaaatggtttcaacaacggggttcatcccgcccatggcatcgcttgaggataaaggcaaacccttcaacagtaaggtcatcatagcaaccaccaacctgtactcgggcttcaccccgaggactatggtgtgccctgatgccctgaaccggaggtttcactttgacatcgacgtgagcgccaaggacgggtacaaaattaacaacaaattggacatcatcaaagcacttgaagatactcacaccaacccagtggcaatgtttcagtacgactgtgcccttctcaacggcatggctgttgaaatgaagagaatgcaacaagatatgttcaagcctcaaccaccccttcagaacgtgtaccaactggttcaagaggtgattgagcgggtggagctccacgagaaggtgtcgagccacccgattttcaaacagatctcaattccttcccaaaaatccgtgttgtacttcctcattgagaaaggacagcacgaggcagcaattgaattctttgagggcatggtgcacgactccatcaaggaggagctccggccgctcatccaacaaacctcatttgtgaaacgcgcttttaagcgcctgaaggaaaactttgagattgttgccctatgtctgaccctcctggccaacatagtgatcatgatccgcgaaactcgcaagagacagaagatggtggacgatgcagtgagtgagtacattgagagagcaaacatcaccaccgacgacaagactcttgatgaggcggaaaagaaccctctggaaaccagcggtgccagcaccgtcggcttcagagagagacctctcccaggccaaaaggcgcgtaatgacgagaactccgagcccgcccagcctgctgaagagcaaccacaagctgaaggaccctacgctggcccgatggagagacagaaaccactgaaagtgaaagcaaaagccccggtcgttaaggaaggaccttacgagggaccggtgaagaagcctgttgctttgaaagtgaaagctaagaacttgatcgtcactgagagtggtgccccaccgaccgacttgcaaaagttggtcatgggcaacaccaagcccgttgagctcatccttgacgggaagacggtagccatttgctgtgctactggagttttcggcactgcttacctcgtgcctcgtcatcttttcgcagaaaagtacgacaagatcatgttggacggcagagccatgacagatagtgactacagagtgtttgagtttgagattaaagtaaaaggacaggacatgctctcagacgctgcgctcatggtgctccaccgtgggaatcgcgtgagagacatcacgaaacactttcgtgacacagcaagaatgaagaaaggcacccccgtcgttggtgtgatcaacaacgccgatgtcgggagactgattttctctggtgaagcccttacctacaaggacattgtagtgtgcatggatggagacaccatgcctgggctctttgcctacaaagccgcaaccaaggctggttattgcggaggagccgtcctcgctaaggacggggctgacacgttcatcgttggcacccactccgctggaggcaatggcgttggatactgctcttgcgtttccaggtccatgcttctcaagatgaaggcacacgttgaccccgaaccacaccacgaggggttgattgttgacaccagagatgtggaagagcgcgttcacgtgatgcgcaaaaccaagcttgcacccaccgttgcgtacggtgtgttccgtcctgagttcgggcctgccgccttgtccaacaaggacccgcgcctgaacgacggtgttgtcctcgacgaagtcatcttctccaaacacaagggagacacaaagatgtctgaggaagacaaagcgctgttccgccgctgtgctgctgactacgcgtcacgcctgcacagcgtgttgggtacggcaaatgccccactgagcatctacgaggcaattaaaggcgttgatggactcgacgcaatggaaccagacaccgcacccggcctcccctgggcactccaggggaagcgccgtggcgcgctcatcgacttcgagaacggcactgttggacccgaagttgaggctgccttgaagctcatggagaaaagagaatacaagtttgcttgccaaaccttcctgaaggacgagattcgcccgatggagaaagtacgtgccggtaagactcgcattgtcgacgtcctacctgttgaacacatcctctacaccaggatgatgattggcagattttgtgcacaaatgcactcaaacaacggaccccaaattggctcggcggtcggttgtaaccctgatgttgattggcaaagatttggcacacacttcgcccaatacagaaacgtgtgggatgtggactattcggccttcgatgctaaccactgcagtgacgccatgaacatcatgtttgaggaagtgtttcgcacagaattcgggttccacccaaacgctgagtggatcctgaagactctcgtgaacacggaacacgcctatgagaacaaacgcatcactgttgaaggcgggatgccatctggttgttccgcaacaagcatcatcaacacaattttgaacaacatctacgtgctctacgctttgcgtagacactatgagggagttgagctggacacttacaccatgatctcttacggagacgatatcgtggtggcaagtgattacgatttggactttgaggctctcaagccccacttcaaatcccttggtcaaaccatcactccagctgacaaaagcgacaaaggttttgttcttggtcactccattactgatgtcactttcctcaaaagacacttccacatggattatggaactgggttttacaaacctgtgatggcctcaaagacccttgaggctatcctctcctttgcacgccgtgggaccatacaggagaagttgatctccgtggcaggactcgctgttcactctggaccagacgagtaccggcgtctcttcgagccctttcaaggcctcttcgagattccaagctacagatcactttacctgcgttgggtgaacgccgtgtgcggcgacgca

SEQ ID NO: 18 - protein, 2109 aa, synthetic construct
  REGION 1..2109: note = Fusion protein: Foot and Mouth Disease Virus (FMDV) and Bovine Rhinovirus Type 2 (BRV2)
GAGQSSPATGSQNQSGNTGSIINNYYMQQYQNSMDTQLGDNAISGGSNEGSTDTTSTHTTNTQNNDWFSKLASSAFTGLFGALLADKKTEETTLLEDRILTTRNGHTTSTTQSSVGVTHGYSTEEDHVAGPNTSGLETRVVQAERFYKKYLFDWTTDKAFGHLEKLELPSDHHGVFGHLVDSYAYMRNGWDVEVSAVGNQFNGGCLLVAMVPEWKEFDTREKYQLTLFPHQFISPRTNMTAHITVPYLGVNRYDQYKKHKPWTLVVMVVSPLTVNNTSAAQIKVYANIAPTYVHVAGELPSKEGIFPVACADGYGGLVTTDPKTADPAYGKVYNPPRTNYPGRFTNLLDVAEACPTFLCFDDGKPYVTTRTDDTRLLAKFDLSLAAKHMSNTYLSGIAQYYTQYSGTINLHFMFTGSTDSKARYMVAYIPPGVETPPDTPERAAHCIHAEWDTGLNSKFTFSIPYVSAADYAYTASDTAETINVQGWVCIYQITHGKAENDTLVVSVSAGKDFELRLPIDPRQQTTATGESADPVTTTVENYGGETQIQRRHHTDIGFIMDRFVKIQSLSPTHVIDLMQTHQHGLVGALLRAATYYFSDLEIVVRHEGNLTWVPNGAPESALLNTSNPTAYNKAPFTRLALPYTAPHRVLATVYNGTSKYAVGGSGRRGDMGSLAARVVKQLPASFNYGAIKADAIHELLVRMKRAELYCPRPLLAIEVSSQDRHKQKIIAPAKQLLNFDLLKLAGDVESNPGPFFFSDVRSNFSKLVDTINQMQEDMSTKHGPDFNRLVSAFEELATGVKAIRTGLDEAKPWYKLIKLLSRLSCMAAVAARSKDPVLVAIMLADTGLEILDSTFVVKKISDSLSSLFHVPAPVFSFGAPILLAGLVKVASSFFRSTPEDLERAEKQLKARDINDIFAILKNGEWLVKLILAIRDWIKAWIASEEKFVTTTDLVPSILEKQQDLNDPSKYKEAKEWLDNARQACLKSGNVHIANLCKVVAPAPSRSRPEPVVVCLRGKSGQGKSFLANVLAQAISTHFTGRTDSVWYCPPDPDHFDGYNQQTVVVMDDLGQNPDGKDFKYFAQMVSTTGFIPPMASLEDKGKPFNSKVIIATTNLYSGFTPRTMVCPDALNRRFHFDIDVSAKDGYKINNKLDIIKALEDTHTNPVAMFQYDCALLNGMAVEMKRMQQDMFKPQPPLQNVYQLVQEVIERVELHEKVSSHPIFKQISIPSQKSVLYFLIEKGQHEAAIEFFEGMVHDSIKEELRPLIQQTSFVKRAFKRLKENFEIVALCLTLLANIVIMIRETRKRQKMVDDAVSEYIERANITTDDKTLDEAEKNPLETSGASTVGFRERPLPGQKARNDENSEPAQPAEEQPQAEGPYAGPMERQKPLKVKAKAPVVKEGPYEGPVKKPVALKVKAKNLIVTESGAPPTDLQKLVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGNGVGYCSCVSRSMLLKMKAHVDPEPHHEGLIVDTRDVEERVHVMRKTKLAPTVAYGVFRPEFGPAALSNKDPRLNDGVVLDEVIFSKHKGDTKMSEEDKALFRRCAADYASRLHSVLGTANAPLSIYEAIKGVDGLDAMEPDTAPGLPWALQGKRRGALIDFENGTVGPEVEAALKLMEKREYKFACQTFLKDEIRPMEKVRAGKTRIVDVLPVEHILYTRMMIGRFCAQMHSNNGPQIGSAVGCNPDVDWQRFGTHFAQYRNVWDVDYSAFDANHCSDAMNIMFEEVFRTEFGFHPNAEWILKTLVNTEHAYENKRITVEGGMPSGCSATSIINTILNNIYVLYALRRHYEGVELDTYTMISYGDDIVVASDYDLDFEALKPHFKSLGQTITPADKSDKGFVLGHSITDVTFLKRHFHMDYGTGFYKPVMASKTLEAILSFARRGTIQEKLISVAGLAVHSGPDEYRRLFEPFQGLFEIPSYRSLYLRWVNAVCGDA

SEQ ID NO: 19 - protein, 40 aa, synthetic construct
  REGION 1..40: note = Foot-and-mouth disease virus
GLIVDTRDVEERVHVMRKTKLAPTVAHGVFNPEFGPAALS

SEQ ID NO: 20 - protein, 40 aa, synthetic construct
  REGION 1..40: note = Foot-and-mouth disease virus
GLIVDTRDVEERVHVMRKTKLAPTVAYGVFRPEFGPAALS

SEQ ID NO: 21 - protein, 24 aa, synthetic construct
  REGION 1..24: note = Foot-and-mouth disease virus
GPYAGPMERQKPLKVRAKAPVVKE

SEQ ID NO: 22 - protein, 24 aa, synthetic construct
  REGION 1..24: note = Foot-and-mouth disease virus
GPYAGPMEPVKVLKVRAKAPVVKE

SEQ ID NO: 23 - DNA, 7589 bp, synthetic construct
  misc_feature 1..7589: note = Fusion nucleotide: Foot and Mouth Disease Virus (FMDV) and Bovine Rhinovirus Type 2 (BRV2)
  source 1..7589: mol_type = other DNA
ttgaaagggggcgctagggtctcacccctagcatgccaacgacagtccccgcgttgcactccacactcacgttgtgcgtgcgcggagctcgatggactatcgttcacccacctacagctggactcacggcaccgtgtggccacttggctggattgtgcggacgaacaccgcttgcgcttctcgcgtgaccggttagtactctcaccaccttccgcccacttggttgttagcgctgtcttgggcactcctgttgggggccgttcgacgctccgcgagtttccccgcacggcaactacggtgatggggccgtaccgcgcgggctgatcgcctggtgtgcttcggctgtcacccgaagcctacctttcaccccccccccccccccccccccccccccccccccccccccctaagttctaccgtcgttcccgacgtaaagggatgtaaccacaagcttactaccgcctttcccggcgttaaagggatgtaaccacaagacttaccttcacccggaagtaaaacggcaacttcacacagttttgcccgttttcatgagaaatgggacgtctgcgcacgaaacgcgccgtcgcttgaggaggacttgtacaaacacgatctaagcaggtttccccaactgacacaaaccgtgcaatttgaaactccgcctgggctttccaggtctagaggggtgacgctttgtactgtgtttgactccacgttcgatccactggcgagtgttagtaacaacactgctgcttcgtagcggagcatgacggccgtgggacccccccccttggtaacaaggacccacggggccaaaagccacgtccgaatggacccgtcatgtgtgcaaacccagcacagtagctttgttgtgaaactcactttaaagtgacattgatactggtactcaagcactggtgacaggctaaggatgcccttcaggtaccccgaggtaacacgtgacactcgggatctgagaaggggaccggggcttctataaaagcgcccggtttaaaaagcttctatgtctgaataggtgaccggaggccggcacctttcttttaattacactggacttatgaacacaactgattgttttatcgctttggtacacgctatcagagagatcagagcatttttcctaccacgagccacaggaatgggggccggccaatccagtccggcaaccgggtcacagaaccaatctggcaacactggaagcatcattaacaactactacatgcaacagtaccagaattccatggacacacagcttggtgacaacgctattagcggaggttccaacgaaggttccacggataccacttccacacacacaaacaacacccaaaacaacgactggttctcgcgcctggcaagttctgcattcagtggtctctttggtgcacttttggctgacaagaagacagaagagacaactctgcttgaagaccgcattctcaccaccaggaacggccacacaacatcgacgacacagtcgagcgttggcgtaacatacggttacgctgtggccgaggacgcggtgtctggacccaatacctcgggtctagagactcgtgttcaacaggcagaacggtttttcaagaaacacctgtttgactggacaccgaacttggcatttggacactgttactacctggaacttcccactgaacacaaaggcgtgtacggcagtctcatgggctcgtacgcctacatgagaaatggatgggacatagaggtgactgctgttggaaaccaattcaacggtggttgtctccttgtcgcgctcgtgccagagctgaaggaactcgacacgcgacagaagtaccagctgaccctctttccccaccagttcatcaacccacgcaccaacatgacggcccacatcaacgtgccgtacgtgggtatcaacaggtacgaccagtacgccctccacaagccgtggacgcttgttgtgatggtggtagccccactcaccgtcaaaactggtggttctgaacagatcaaggtttacatgaatgcagcgccaacctacgtgcatgtggcgggagagctgccctcgaaagagggaatagttcccgtcgcgtgtgcggacggttacggcaacatggtgaccacggacccgaagacggccgatccagtttacgggaaagtgttcaacccccccaggacaaacctccctgggcgcttcacgaacttccttgatgttgcggaggcatgtccaactttcctccgctttggagaagtaccatttgtgaagacggtgaactctggtgaccgcttgctggccaagttcgacgtgtccctcgctgcagggcacatgtccaacacctacttggctggcctggcgcagtactacacacagtacagcggcaccatgaacgtccacttcatgttcaccgggcccacggatgctaaagcccgatacatggtggcttatgtcccccctggcatgacaccgcccacggaccctgagcacgccgcacactgcattcactctgagtgggatactggtcttaactctaagtttaccttttccataccttacctctctgctgctgactatgcctacactgcttctgacgtggcggagaccacgagtgtgcagggatgggtgtgtatctatcagatcacccacggcaaggctgagggagacgcactggtcgtttctgtcagcgccggcaaagactttgagtttcgcttgcctgttgacgcacgccagcaaaccaccaccactggcgaatcagcagatccagtcacaaccacggttgagaactatggaggagagactcagacagccagacggcttcacactgacgtcgccttcattcttgacaggtttgtgaaactcactgctcccaagaacatccaaaccctcgatctcatgcagatcccctcacacacgctggttggagcactacttcgttctgcgacgtactacttctcagacctggaggtcgcgcttgtccacacaggcccggtcacctgggtgcccaacggcgcgcccaaggatgctctaaacaaccagaccaacccaactgcctatcagaagcaacccatcacccgcctggcactcccctacaccgccccccatcgtgtgctggcaacagtgtacaacgggaagacggcgtacggggaaacgacctcaaggcgcggcgacatggcggccctcgcacaaaggttgagcgctcggctgcccacctccttcaactacggcgccgtgaaggccgacaccatcactgagcttttgatccgcatgaagcgcgcggagacatattgccctaggcctttactagcccttgacaccactcaggaccgccgcaaacaggagatcattgcacctgagaagcagcttctgaattttgacctgcttaagctagccggagacgttgagtccaaccctgggcccttcttcttctccgacgttaggtcaaacttttccaagctggtagacacaatcaaccagatgcaggaagacatgtccacaaagcacggacctgactttaaccggttggtgtccgcttttgaggagttggccactggagtgaaagccatcaggaccggtcttgacgaggccaagccctggtacaagcttatcaagctcctgagccgcctgtcgtgcatggccgctgtggcagcacggtcaaaggacccagtccttgtggccatcatgctggctgacaccggtctcgagattctggacagcaccttcgtcgtgaagaagatctccgactcgctctccagtctcttccacgtgccggcccccgtcttcagtttcggagccccgattctgttagccgggttggtcaaggtcgcctcgagtttcttccggtccacgcccgaagaccttgagagagcagagaaacagctcaaagcacgtgacatcaacgacattttcgccattctcaagaacggcgagtggctggtcaaattgatccttgccatccgcgactggatcaaggcatggatagcctcagaagaaaagtttgtcaccacgacagacttggtacctagcatccttgaaaaacagcaggacctcaacgacccaagcaagtacaaggaagccaaggagtggctcgacaacgcgcgccaagcgtgtttgaagagcgggaacgtccacattgccaacctgtgcaaagtggtcgccccggcacccagcaggtcgagacccgagcccgtggtcgtttgcctccgtggcaagtccggtcagggcaagagtttccttgcaaacgtgctcgcacaagcaatctctacccatttcactggcaggaccgattcagtttggtactgcccgcctgaccctgaccacttcgacggttacaaccaacagactgtcgttgtgatggacgatttgggccagaaccccgacggcaaagacttcaagtacttcgcccaaatggtttcaacaacggggttcatcccgcccatggcatcgcttgaggataaaggcaaacccttcaacagtaaggtcatcatagcaaccaccaacctgtactcgggcttcaccccgaggactatggtgtgccctgatgccctgaaccggaggtttcactttgacatcgacgtgagcgccaaggacgggtacaaaattaacaacaaattggacatcatcaaagcacttgaagatactcacaccaacccagtggcaatgtttcagtacgactgtgcccttctcaacggcatggctgttgaaatgaagagaatgcaacaagatatgttcaagcctcaaccaccccttcagaacgtgtaccaactggttcaagaggtgattgagcgggtggagctccacgagaaggtgtcgagccacccgattttcaaacagatctcaattccttcccaaaaatccgtgttgtacttcctcattgagaaaggacagcacgaggcagcaattgaattctttgagggcatggtgcacgactccatcaaggaggagctccggccgctcatccaacaaacctcatttgtgaaacgcgcttttaagcgcctgaaggaaaactttgagattgttgccctatgtctgaccctcctggccaacatagtgatcatgatccgcgaaactcgcaagagacagaagatggtggacgatgcagtgagtgagtacattgagagagcaaacatcaccaccgacgacaagactcttgatgaggcggaaaagaaccctctggaaaccagcggtgccagcaccgtcggcttcagagagagacctctcccaggccaaaaggcgcgtaatgacgagaactccgagcccgcccagcctgctgaagagcaaccacaagctgaaggaccctacgctggcccgatggagagaccagttaaagttaaagtgaaagcaaaagccccggtcgttaaggaaggaccttacgagggaccggtgaagaagcctgttgctttgaaagtgaaagctaagaacttgatcgtcactgagagtggtgccccaccgaccgacttgcaaaagttggtcatgggcaacaccaagcccgttgagctcatccttgacgggaagacggtagccatttgctgtgctactggagttttcggcactgcttacctcgtgcctcgtcatcttttcgcagaaaagtacgacaagatcatgttggacggcagagccatgacagatagtgactacagagtgtttgagtttgagattaaagtaaaaggacaggacatgctctcagacgctgcgctcatggtgctccaccgtgggaatcgcgtgagagacatcacgaaacactttcgtgacacagcaagaatgaagaaaggcacccccgtcgttggtgtgatcaacaacgccgatgtcgggagactgattttctctggtgaagcccttacctacaaggacattgtagtgtgcatggatggagacaccatgcctgggctctttgcctacaaagccgcaaccaaggctggttattgcggaggagccgtcctcgctaaggacggggctgacacgttcatcgttggcacccactccgctggaggcaatggcgttggatactgctcttgcgtttccaggtccatgcttctcaagatgaaggcacacgttgaccccgaaccacaccacgaggggttgattgttgacaccagagatgtggaagagcgcgttcacgtgatgcgcaaaaccaagcttgcacccaccgttgcgtacggtgtgttccgtcctgagttcgggcctgccgccttgtccaacaaggacccgcgcctgaacgacggtgttgtcctcgacgaagtcatcttctccaaacacaagggagacacaaagatgtctgaggaagacaaagcgctgttccgccgctgtgctgctgactacgcgtcacgcctgcacagcgtgttgggtacggcaaatgccccactgagcatctacgaggcaattaaaggcgttgatggactcgacgcaatggaaccagacaccgcacccggcctcccctgggcactccaggggaagcgccgtggcgcgctcatcgacttcgagaacggcactgttggacccgaagttgaggctgccttgaagctcatggagaaaagagaatacaagtttgcttgccaaaccttcctgaaggacgagattcgcccgatggagaaagtacgtgccggtaagactcgcattgtcgacgtcctacctgttgaacacatcctctacaccaggatgatgattggcagattttgtgcacaaatgcactcaaacaacggaccccaaattggctcggcggtcggttgtaaccctgatgttgattggcaaagatttggcacacacttcgcccaatacagaaacgtgtgggatgtggactattcggccttcgatgctaaccactgcagtgacgccatgaacatcatgtttgaggaagtgtttcgcacagaattcgggttccacccaaacgctgagtggatcctgaagactctcgtgaacacggaacacgcctatgagaacaaacgcatcactgttgaaggcgggatgccatctggttgttccgcaacaagcatcatcaacacaattttgaacaacatctacgtgctctacgctttgcgtagacactatgagggagttgagctggacacttacaccatgatctcttacggagacgatatcgtggtggcaagtgattacgatttggactttgaggctctcaagccccacttcaaatcccttggtcaaaccatcactccagctgacaaaagcgacaaaggttttgttcttggtcactccattactgatgtcactttcctcaaaagacacttccacatggattatggaactgggttttacaaacctgtgatggcctcaaagacccttgaggctatcctctcctttgcacgccgtgggaccatacaggagaagttgatctccgtggcaggactcgctgttcactctggaccagacgagtaccggcgtctcttcgagccctttcaaggcctcttcgagattccaagctacagatcactttacctgcgttgggtgaacgccgtgtgcggcgacgcataatccctcagagactacattggcatactgtttctgaggcgcgcgacgccgtaggagtgaaaagcctgaaagggcttttcccgcttcctattccaaaaaaaaaaaaaaaaaa

SEQ ID NO: 24 - DNA, 7600 bp, synthetic construct
  misc_feature 1..7600: note = Fusion nucleotide: Foot and Mouth Disease Virus (FMDV) and Bovine Rhinovirus Type 2 (BRV2)
  source 1..7600: mol_type = other DNA
ttgaaagggggcgctagggtctcacccctagcatgccaacgacagtccccgcgttgcactccacactcacgttgtgcgtgcgcggagctcgatggactatcgttcacccacctacagctggactcacggcaccgtgtggccacttggctggattgtgcggacgaacaccgcttgcgcttctcgcgtgaccggttagtactctcaccaccttccgcccacttggttgttagcgctgtcttgggcactcctgttgggggccgttcgacgctccgcgagtttccccgcacggcaactacggtgatggggccgtaccgcgcgggctgatcgcctggtgtgcttcggctgtcacccgaagcctacctttcaccccccccccccccccccccccccccccccccccccccccctaagttctaccgtcgttcccgacgtaaagggatgtaaccacaagcttactaccgcctttcccggcgttaaagggatgtaaccacaagacttaccttcacccggaagtaaaacggcaacttcacacagttttgcccgttttcatgagaaatgggacgtctgcgcacgaaacgcgccgtcgcttgaggaggacttgtacaaacacgatctaagcaggtttccccaactgacacaaaccgtgcaatttgaaactccgcctgggctttccaggtctagaggggtgacgctttgtactgtgtttgactccacgttcgatccactggcgagtgttagtaacaacactgctgcttcgtagcggagcatgacggccgtgggacccccccccttggtaacaaggacccacggggccaaaagccacgtccgaatggacccgtcatgtgtgcaaacccagcacagtagctttgttgtgaaactcactttaaagtgacattgatactggtactcaagcactggtgacaggctaaggatgcccttcaggtaccccgaggtaacacgtgacactcgggatctgagaaggggaccggggcttctataaaagcgcccggtttaaaaagcttctatgtctgaataggtgaccggaggccggcacctttcttttaattacactggacttatgaacacaactgattgttttatcgctttggtacacgctatcagagagatcagagcatttttcctaccacgagccacaggaatgggggccggccaatccagtccggcaaccgggtcacaaaaccaatcaggcaacactggtagtatcatcaacaactactacatgcagcagtaccagaactccatggatacacaacttggcgacaacgccattagcggtggttccaacgagggctccactgacactacctccacacacacaaccaacacacagaacaatgactggttttcaaagctggccagttctgccttcagcggtctcttcggcgctcttctcgctgacaaaaagacagaggagactaccctcctggaggaccgcatccttaccacccgcaacggacacaccacctcgacaacccagtcgagtgtgggtgtcacctacgggtactccactggtgaagaccacgtctctggacctaacacatctggcctggagacgcgagtggtacaggcagagagattcttcaagaaacacttgtttgattggacaactgataaagcttttggacacctggaaaaactggaactccccaccgaacacaagggtgtctacgggcacttggtggactctttcgcatacatgagaaatggctgggacgtggaggtgaccgccgttggcaaccagttcaacggtgggtgtctcctggtggccatggtacctgagtggaaagagtttacccctcgtgagaaataccagctcaccctgtttccacaccaatttatcaaccccagaaccaacatgacagcccacatcacggtcccgtaccttggtgtcaataggtatgaccagtacaaacagcacaaaccctggacactggtcgtgatggtggtttcgccactgaccaccagcagcattggggcctcacagattaaggtctacgccaacattgccccaaccttcgttcacgtggccggcgagctcccatcgaaagaagggatcgtgccggttgcttgtacagacgggtacggtggcctggtgacaacagacccgaaaacagctgaccctgtttatggtatggtgtacaacccgcccagaaccaactaccctgggcgctttacaaacttgttggacgtggccgaggcttgcccgaccttcctctgttttgacgacgggaaaccgtacgttgtgacaaggacggacgaccaacgcctcctggccaagtttgacgtttcccttgctgcaaagcacatgtcaaacacctacctctcagggatagcacagtactacacgcagtactctggcactatcaatctgcatttcatgttcactggctctactgaatcaaaggcccggtacatggtggcgtacattccacctggcatggacaacccaccggacacacctgagaaggctgcacattgcatccacgccgagtgggacaccgggctgaactccaaatttactttttctatcccgtacgtgtctgctgcagactacgcatacactgcgtctgacgtggcagaaacaacaaacgtacaggggtgggtctgcatataccaaatcactcacgggaaggctgaacaggacactctggtcgtgtcggtcagcgccggcaaggactttgaactgcgcctcccaattgacccccgcacgcaaaccaccactgccggggagtcagcagaccctgtcaccaccaccgttgagaactacggtggtgagacacaggctcagcgacgtcagcacactgacgtcggcttcatcatggacaggtttgcgaaaatcagccccgtgagccccacgcacgtcattgacctcatgcaaacacaccaacacgcgttggtgggtgcccttttgcgtgcagccacgtactacttctccgatctggagattgtggtgcgtcatgatggcaacttgacgtgggtgcccaatggagcacctgtagaagccttggccaacacaagcaaccccaccgcctaccacaagcagccatttacgagacttgcgctcccttacaccgcgccgcaccgagtgttggcaacagtgtataacggagtaagcaagtactctacaactggtaatggcagaaggggtgacctggggcctcttgcggcgcgggtcgccgcacagctccccagctctttcaattttggtgcaattcgggccacgaccgtccacgagcttctcgtgcgcatgaaacgtgccgagctctactgtcccaggcctctgctggcagtggaagtgttgtcgcaggacagacacaagcaaaagatcattgcacctgcaaagcaacttctgaattttgacctgcttaagctagccggagacgttgagtccaaccctgggcccttcttcttctccgacgttaggtcaaacttttccaagctggtagacacaatcaaccagatgcaggaagacatgtccacaaagcacggacctgactttaaccggttggtgtccgcttttgaggagttggccactggagtgaaagccatcaggaccggtcttgacgaggccaagccctggtacaagcttatcaagctcctgagccgcctgtcgtgcatggccgctgtggcagcacggtcaaaggacccagtccttgtggccatcatgctggctgacaccggtctcgagattctggacagcaccttcgtcgtgaagaagatctccgactcgctctccagtctcttccacgtgccggcccccgtcttcagtttcggagccccgattctgttagccgggttggtcaaggtcgcctcgagtttcttccggtccacgcccgaagaccttgagagagcagagaaacagctcaaagcacgtgacatcaacgacattttcgccattctcaagaacggcgagtggctggtcaaattgatccttgccatccgcgactggatcaaggcatggatagcctcagaagaaaagtttgtcaccacgacagacttggtacctagcatccttgaaaaacagcaggacctcaacgacccaagcaagtacaaggaagccaaggagtggctcgacaacgcgcgccaagcgtgtttgaagagcgggaacgtccacattgccaacctgtgcaaagtggtcgccccggcacccagcaggtcgagacccgagcccgtggtcgtttgcctccgtggcaagtccggtcagggcaagagtttccttgcaaacgtgctcgcacaagcaatctctacccatttcactggcaggaccgattcagtttggtactgcccgcctgaccctgaccacttcgacggttacaaccaacagactgtcgttgtgatggacgatttgggccagaaccccgacggcaaagacttcaagtacttcgcccaaatggtttcaacaacggggttcatcccgcccatggcatcgcttgaggataaaggcaaacccttcaacagtaaggtcatcatagcaaccaccaacctgtactcgggcttcaccccgaggactatggtgtgccctgatgccctgaaccggaggtttcactttgacatcgacgtgagcgccaaggacgggtacaaaattaacaacaaattggacatcatcaaagcacttgaagatactcacaccaacccagtggcaatgtttcagtacgactgtgcccttctcaacggcatggctgttgaaatgaagagaatgcaacaagatatgttcaagcctcaaccaccccttcagaacgtgtaccaactggttcaagaggtgattgagcgggtggagctccacgagaaggtgtcgagccacccgattttcaaacagatctcaattccttcccaaaaatccgtgttgtacttcctcattgagaaaggacagcacgaggcagcaattgaattctttgagggcatggtgcacgactccatcaaggaggagctccggccgctcatccaacaaacctcatttgtgaaacgcgcttttaagcgcctgaaggaaaactttgagattgttgccctatgtctgaccctcctggccaacatagtgatcatgatccgcgaaactcgcaagagacagcagatggtggacgatgcagtgagtgagtacattgagagagcaaacatcaccaccgacgacaagactcttgatgaggcggaaaagaaccctctggaaaccagcggtgccagcaccgtcggcttcagagagagacctctcccaggccaaaaggcgcgtaatgacgagaactccgagcccgcccagcctgctgaagagcaaccacaagctgaaggaccctacgctggcccgatggagagaccagttaaagttaaagtgaaagcaaaagccccggtcgttaaggaaggaccttacgagggaccggtgaagaagcctgttgctttgaaagtgaaagctaagaacttgatcgtcactgagagtggtgccccaccgaccgacttgcaaaagttggtcatgggcaacaccaagcccgttgagctcatccttgacgggaagacggtagccatttgctgtgctactggagttttcggcactgcttacctcgtgcctcgtcatcttttcgcagaaaagtacgacaagatcatgttggacggcagagccatgacagatagtgactacagagtgtttgagtttgagattaaagtaaaaggacaggacatgctctcagacgctgcgctcatggtgctccaccgtgggaatcgcgtgagagacatcacgaaacactttcgtgacacagcaagaatgaagaaaggcacccccgtcgttggtgtgatcaacaacgccgatgtcgggagactgattttctctggtgaagcccttacctacaaggacattgtagtgtgcatggatggagacaccatgcctgggctctttgcctacaaagccgcaaccaaggctggttattgcggaggagccgtcctcgctaaggacggggctgacacgttcatcgttggcacccactccgctggaggcaatggcgttggatactgctcttgcgtttccaggtccatgcttctcaagatgaaggcacacgttgaccccgaaccacaccacgaggggttgattgttgacaccagagatgtggaagagcgcgttcacgtgatgcgcaaaaccaagcttgcacccaccgttgcgtacggtgtgttccgtcctgagttcgggcctgccgccttgtccaacaaggacccgcgcctgaacgacggtgttgtcctcgacgaagtcatcttctccaaacacaagggagacacaaagatgtctgaggaagacaaagcgctgttccgccgctgtgctgctgactacgcgtcacgcctgcacagcgtgttgggtacggcaaatgccccactgagcatctacgaggcaattaaaggcgttgatggactcgacgcaatggaaccagacaccgcacccggcctcccctgggcactccaggggaagcgccgtggcgcgctcatcgacttcgagaacggcactgttggacccgaagttgaggctgccttgaagctcatggagaaaagagaatacaagtttgcttgccaaaccttcctgaaggacgagattcgcccgatggagaaagtacgtgccggtaagactcgcattgtcgacgtcctacctgttgaacacatcctctacaccaggatgatgattggcagattttgtgcacaaatgcactcaaacaacggaccccaaattggctcggcggtcggttgtaaccctgatgttgattggcaaagatttggcacacacttcgcccaatacagaaacgtgtgggatgtggactattcggccttcgatgctaaccactgcagtgacgccatgaacatcatgtttgaggaagtgtttcgcacagaattcgggttccacccaaacgctgagtggatcctgaagactctcgtgaacacggaacacgcctatgagaacaaacgcatcactgttgaaggcgggatgccatctggttgttccgcaacaagcatcatcaacacaattttgaacaacatctacgtgctctacgctttgcgtagacactatgagggagttgagctggacacttacaccatgatctcttacggagacgatatcgtggtggcaagtgattacgatttggactttgaggctctcaagccccacttcaaatcccttggtcaaaccatcactccagctgacaaaagcgacaaaggttttgttcttggtcactccattactgatgtcactttcctcaaaagacacttccacatggattatggaactgggttttacaaacctgtgatggcctcaaagacccttgaggctatcctctcctttgcacgccgtgggaccatacaggagaagttgatctccgtggcaggactcgctgttcactctggaccagacgagtaccggcgtctcttcgagccctttcaaggcctcttcgagattccaagctacagatcactttacctgcgttgggtgaacgccgtgtgcggcgacgcataatccctcagagactacattggcatactgtttctgaggcgcgcgacgccgtaggagtgaaaagcctgaaagggcttttcccgcttcctattccaaaaaaaaaaaaaaaaa

SEQ ID NO: 25 - DNA, 7597 bp, synthetic construct
  misc_feature 1..7597: note = Fusion Nucleotide Sequence containing O1 Campos strain of FMD (complete genome)
  source 1..7597: mol_type = other DNA
ttgaaagggggcgctagggtctcacccctagcatgccaacgacagtccccgcgttgcactccacactcacgttgtgcgtgcgcggagctcgatggactatcgttcacccacctacagctggactcacggcaccgtgtggccacttggctggattgtgcggacgaacaccgcttgcgcttctcgcgtgaccggttagtactctcaccaccttccgcccacttggttgttagcgctgtcttgggcactcctgttgggggccgttcgacgctccgcgagtttccccgcacggcaactacggtgatggggccgtaccgcgcgggctgatcgcctggtgtgcttcggctgtcacccgaagcctacctttcaccccccccccccccccccccccccccccccccccccccccctaagttctaccgtcgttcccgacgtaaagggatgtaaccacaagcttactaccgcctttcccggcgttaaagggatgtaaccacaagacttaccttcacccggaagtaaaacggcaacttcacacagttttgcccgttttcatgagaaatgggacgtctgcgcacgaaacgcgccgtcgcttgaggaggacttgtacaaacacgatctaagcaggtttccccaactgacacaaaccgtgcaatttgaaactccgcctgggctttccaggtctagaggggtgacactttgtactgtgtttgactccacgttcgatccactggcgagtgttagtaacaacactgctgcttcgtagcggagcatgacggccgtgggacccccccccttggtaacaaggacccacggggccaaaagccacgtccgaatggacccgtcatgtgtgcaaacccagcacagtagctttgttgtgaaactcactttaaagtgacattgatactggtactcaagcactggtgacaggctaaggatgcccttcaggtaccccgaggtaacacgtgacactcgggatctgagaaggggaccggggcttctataaaagcgcccggtttaaaaagcttctatgtctgaataggtgaccggaggccggcacctttcttttaattacactggacttatgaacacaactgattgttttatcgctttggtacacgctatcagagagatcagagcatttttcctaccacgagccacaggaatgggggccggccaatccagtccggcgaccggctcgcagaaccaatctggcaacactggcagcataattaacaactactacatgcagcaataccagaactccatggacacacagcttggtgacaacgcaatcagtggaggctctaacgagggctccaccgacacaacctccacccacacaaccaacacccagaacaatgactggttctccaaacttgcaagctctgctttcagcggtcttttcggcgctcttctcgccgacaagaagacagaggagaccactctcctcgaagaccgcatcctcaccacccgtaacggccacaccacgtcgacaacccagtcaagcgttggagtcacatacgggtacgcaacagctgaggattttgtgagcggaccgaacacttccggtctcgagaccagagttgtgcaggcagaacggtttttcaaaacccacctcttcgactgggtcaccagtgactcattcggacgttgccacctcctggaactcccgaccgaccacaaaggtgtctacggcagcctgaccgactcgtatgcatatatgagaaacggctgggatgtcgaggtcaccgcggttggcaaccagttcaacggagggtgcctgctggtcgcaatggtaccagagcttcgttctatccaaaagagggaactgtaccagctcacacttttccctcaccagttcatcaacccacgcacgaacatgactgcgcacatcacagtgccctttgttggcgtcaaccgctacgaccagtacaaggttcacaagccttggacccttgtggttatggttgtagcccctctgaccgtcaacactgaaggtgcccctcagatcaaggtgtatgccaacattgccccaaccaacgtgcacgtcgcgggtgagtttccttccaaggagggaatattccccgtggcctgtagcgacggctatggtggcctggtgaccacggacccgaagacggctgaccccgtttatgggaaagtgttcaaccccccccgcaaccagttgccggggcgttttaccaacctccttgatgtggctgaggcatgcccgacgtttctgcacttcgagggtgacgtaccgtacgtgaccacgaaaacagactcggacagggtgcttgctcagtttgatatgtctttggcagcaaaacacatgtcaaacaccttcctcgcaggtcttgcgcagtactacacacagtacagtggcaccatcaacctgcacttcatgttcacaggacccactgacgcgaaggcgcgttacatgattgcctacgccccaccaggcatggagccgcccaagacacctgaggcggccgcgcactgcattcatgctgaatgggacactgggttgaactcaaagtttactttttccatcccctacctctcggccgccgattacgcgtacaccgcgtctgacgtggccgagaccacaaatgtgcagggatgggtctgcttgtttcaaattacacatggcaaggccgacggcgacgctctggtcgtactggctagtgctggtaaagactttgagctaaggctgccggtggacgcccgtgcggaaaccacttctgcgggcgagtcagcggatcctgtcaccgccactgttgaaaactacggtggcgaaacacagatccagaggcgccaacacacggacgtctcgttcatcatggacagatttgtgaaagtgacaccgcaaaaccaaattaacattttggacctcatgcagattccatcacacactttggtgggagcgctcctacgcgcgtccacttactacttctctgacttggagatagcagtaaaacacgagggagacctcacctgggttccaaatggagcgcctgaaaaggcgttggacaacaccaccaacccaactgcttaccacaaggcaccactcacccggcttgccctgccctacaccgcgccccaccgcgtgttggcaaccgtgtacaacggtgagtgcaggtacagcagaaatgctgtgcccaacgtgagaggtgaccttcaggtgttggctcaaaaggtggcacggacgctgcctacctccttcaactacggtgccatcaaagcgacccgggtcaccgagttgctttaccggatgaagagggccgaaacatactgtccaaggcccttgctggcaatccacccaactgaagccagacacaaacagaaaattgtggcaccggtgaaacagttctgaattttgaccttctcaagctagccggagacgttgagtccaaccctgggcccttcttcttctccgacgttaggtcaaacttttccaagctggtagacacaatcaaccagatgcaggaagacatgtccacaaagcacggacctgactttaaccggttggtgtccgcttttgaggagttggccactggagtgaaagccatcaggaccggtcttgacgaggccaagccctggtacaagcttatcaagctcctgagccgcctgtcgtgcatggccgctgtggcagcacggtcaaaggacccagtccttgtggccatcatgctggctgacaccggtctcgagattctggacagcaccttcgtcgtgaagaagatctccgactcgctctccagtctcttccacgtgccggcccccgtcttcagtttcggagccccgattctgttagccgggttggtcaaggtcgcctcgagtttcttccggtccacgcccgaagaccttgagagagcagagaaacagctcaaagcacgtgacatcaacgacattttcgccattctcaagaacggcgagtggctggtcaaattgatccttgccatccgcgactggatcaaggcatggatagcctcagaagaaaagtttgtcaccacgacagacttggtacctagcatccttgaaaaacagcaggacctcaacgacccaagcaagtacaaggaagccaaggagtggctcgacaacgcgcgccaagcgtgtttgaagagcgggaacgtccacattgccaacctgtgcaaagtggtcgccccggcacccagcaggtcgagacccgagcccgtggtcgtttgcctccgtggcaagtccggtcagggcaagagtttccttgcaaacgtgctcgcacaagcaatctctacccatttcactggcaggaccgattcagtttggtactgcccgcctgaccctgaccacttcgacggttacaaccaacagactgtcgttgtgatggacgatttgggccagaaccccgacggcaaagacttcaagtacttcgcccaaatggtttcaacaacggggttcatcccgcccatggcatcgcttgaggataaaggcaaacccttcaacagtaaggtcatcatagcaaccaccaacctgtactcgggcttcaccccgaggactatggtgtgccctgatgccctgaaccggaggtttcactttgacatcgacgtgagcgccaaggacgggtacaaaattaacaacaaattggacatcatcaaagcacttgaagatactcacaccaacccagtggcaatgtttcagtacgactgtgcccttctcaacggcatggctgttgaaatgaagagaatgcaacaagatatgttcaagcctcaaccaccccttcagaacgtgtaccaactggttcaagaggtgattgagcgggtggagctccacgagaaggtgtcgagccacccgattttcaaacagatctcaattccttcccaaaaatccgtgttgtacttcctcattgagaaaggacagcacgaggcagcaattgaattctttgagggcatggtgcacgactccatcaaggaggagctccggccgctcatccaacaaacctcatttgtgaaacgcgcttttaagcgcctgaaggaaaactttgagattgttgccctatgtctgaccctcctggccaacatagtgatcatgatccgcgaaactcgcaagagacagaagatggtggacgatgcagtgagtgagtacattgagagagcaaacatcaccaccgacgacaagactcttgatgaggcggaaaagaaccctctggaaaccagcggtgccagcaccgtcggcttcagagagagacctctcccaggccaaaaggcgcgtaatgacgagaactccgagcccgcccagcctgctgaagagcaaccacaagctgaaggaccctacgctggcccgatggagagaccagttaaagttaaagtgaaagcaaaagccccggtcgttaaggaaggaccttacgagggaccggtgaagaagcctgttgctttgaaagtgaaagctaagaacttgatcgtcactgagagtggtgccccaccgaccgacttgcaaaagttggtcatgggcaacaccaagcccgttgagctcatccttgacgggaagacggtagccatttgctgtgctactggagttttcggcactgcttacctcgtgcctcgtcatcttttcgcagaaaagtacgacaagatcatgttggacggcagagccatgacagatagtgactacagagtgtttgagtttgagattaaagtaaaaggacaggacatgctctcagacgctgcgctcatggtgctccaccgtgggaatcgcgtgagagacatcacgaaacactttcgtgacacagcaagaatgaagaaaggcacccccgtcgttggtgtgatcaacaacgccgatgtcgggagactgattttctctggtgaagcccttacctacaaggacattgtagtgtgcatggatggagacaccatgcctgggctctttgcctacaaagccgcaaccaaggctggttattgcggaggagccgtcctcgctaaggacggggctgacacgttcatcgttggcacccactccgctggaggcaatggcgttggatactgctcttgcgtttccaggtccatgcttctcaagatgaaggcacacgttgaccccgaaccacaccacgaggggttgattgttgacaccagagatgtggaagagcgcgttcacgtgatgcgcaaaaccaagcttgcacccaccgttgcgtacggtgtgttccgtcctgagttcgggcctgccgccttgtccaacaaggacccgcgcctgaacgacggtgttgtcctcgacgaagtcatcttctccaaacacaagggagacacaaagatgtctgaggaagacaaagcgctgttccgccgctgtgctgctgactacgcgtcacgcctgcacagcgtgttgggtacggcaaatgccccactgagcatctacgaggcaattaaaggcgttgatggactcgacgcaatggaaccagacaccgcacccggcctcccctgggcactccaggggaagcgccgtggcgcgctcatcgacttcgagaacggcactgttggacccgaagttgaggctgccttgaagctcatggagaaaagagaatacaagtttgcttgccaaaccttcctgaaggacgagattcgcccgatggagaaagtacgtgccggtaagactcgcattgtcgacgtcctacctgttgaacacatcctctacaccaggatgatgattggcagattttgtgcacaaatgcactcaaacaacggaccccaaattggctcggcggtcggttgtaaccctgatgttgattggcaaagatttggcacacacttcgcccaatacagaaacgtgtgggatgtggactattcggccttcgatgctaaccactgcagtgacgccatgaacatcatgtttgaggaagtgtttcgcacagaattcgggttccacccaaacgctgagtggatcctgaagactctcgtgaacacggaacacgcctatgagaacaaacgcatcactgttgaaggcgggatgccatctggttgttccgcaacaagcatcatcaacacaattttgaacaacatctacgtgctctacgctttgcgtagacactatgagggagttgagctggacacttacaccatgatctcttacggagacgatatcgtggtggcaagtgattacgatttggactttgaggctctcaagccccacttcaaatcccttggtcaaaccatcactccagctgacaaaagcgacaaaggttttgttcttggtcactccattactgatgtcactttcctcaaaagacacttccacatggattatggaactgggttttacaaacctgtgatggcctcaaagacccttgaggctatcctctcctttgcacgccgtgggaccatacaggagaagttgatctccgtggcaggactcgctgttcactctggaccagacgagtaccggcgtctcttcgagccctttcaaggcctcttcgagattccaagctacagatcactttacctgcgttgggtgaacgccgtgtgcggcgacgcataatccctcagagactacattggcatactgtttctgaggcgcgcgacgccgtaggagtgaaaagcctgaaagggcttttcccgcttcctattccaaaaaaaaaaaaaaaaaa

SEQ ID NO: 26 - DNA, 7586 bp, synthetic construct
  misc_feature 1..7586: note = Fusion Nucleotide Sequence containing C3 Indaial strain of FMD (complete genome)
  source 1..7586: mol_type = other DNA
ttgaaagggggcgctagggtctcacccctagcatgccaacgacagtccccgcgttgcactccacactcacgttgtgcgtgcgcggagctcgatggactatcgttcacccacctacagctggactcacggcaccgtgtggccacttggctggattgtgcggacgaacaccgcttgcgcttctcgcgtgaccggttagtactctcaccaccttccgcccacttggttgttagcgctgtcttgggcactcctgttgggggccgttcgacgctccgcgagtttccccgcacggcaactacggtgatggggccgtaccgcgcgggctgatcgcctggtgtgcttcggctgtcacccgaagcctacctttcaccccccccccccccccccccccccccccccccccccccccctaagttctaccgtcgttcccgacgtaaagggatgtaaccacaagcttactaccgcctttcccggcgttaaagggatgtaaccacaagacttaccttcacccggaagtaaaacggcaacttcacacagttttgcccgttttcatgagaaatgggacgtctgcgcacgaaacgcgccgtcgcttgaggaggacttgtacaaacacgatctaagcaggtttccccaactgacacaaaccgtgcaatttgaaactccgcctgggctttccaggtctagaggggtgacactttgtactgtgtttgactccacgttcgatccactggcgagtgttagtaacaacactgctgcttcgtagcggagcatgacggccgtgggacccccccccttggtaacaaggacccacggggccaaaagccacgtccgaatggacccgtcatgtgtgcaaacccagcacagtagctttgttgtgaaactcactttaaagtgacattgatactggtactcaagcactggtgacaggctaaggatgcccttcaggtaccccgaggtaacacgtgacactcgggatctgagaaggggaccggggcttctataaaagcgcccggtttaaaaagcttctatgtctgaataggtgaccggaggccggcacctttcttttaattacactggacttatgaacacaactgattgttttatcgctttggtacacgctatcagagagatcagagcatttttcctaccacgagccacaggaatgggagccggacaatccagcccggcgactggctcgcagaaccaatctggcaacactggtagcataatcaacaactactacatgcaacagtaccaaaattccatggacacacagctgggtgacaatgctattagtggtggctccaacgagggctccacagatacaacttccacccacacaaccaacactcaaaacaacgactggttttccaaactcgccagttctgcctttagcggtcttttcggtgctcttcttgccgacaagaagaccgaggaaaccacactacttgaagaccgcatcctcaccacccgcaacggccacacgacgtcgacaactcagtcgagcgttggggtcacatacgggtacgcaacaactgaggatagcacgtcagggcccaacacatccggccttgagacacgtgttcaccaggcagaacggtttttcaagatgacactctttgaatgggttccctcccagagttttggacacatgcacaaggtcgttctgccctcagaaccgaaaggtgtctatgggggtctcgtcaagtcatacgcgtacatgcgcaatggctgggacgttgaggtgactgctgttggaaaccagttcaacggcggttgtctcctggtggcgctcgttcctgaaatgggtgacatcagtgacagagagaagtaccaactgactctctacccccaccaattcatcaacccacgcactaacatgacggcacacatcaccgtgccttacgtgggtgtcaacagatacgaccaatacaaccaacacaagccctggactcttgtcgtcatggtcgttgctccacttactgtgaacacatcaggtgcccagcagatcaaggtgtatgccaacatagccccaaccaacgttcacgttgctggtgaacttccctccaaggaggggatcttccccgttgcgtgtgccgacggctatggcaacatggtgacaactgacccgaagacagctgaccctgcctacgggaaagtctacaatccacccaggaccgccctgccgggccggttcacaaactacctggatgttgctgaggcttgccccactctcctgacgttcgagaacgtgccttacgtttcaacacggactgatggacaaaggctgttggccaagttcgacgtgtcattggcagcgaaacacatgtcaaacacttacttggctggcttggcccagtactacacacagtacgctgggacaatcaacctgcacttcatgttcactgggccaaccgacgcgaaagctcggtacatggtggcatacgtgccccctggcatggaagcaccagacaacccagaggaggctgcccactgcatacacgcagagtgggacactggtttgaactctaagttcacattttcaatcccgtacatctcggccgctgactacgcatacaccgcgtccagcgaggctgaaacaacaagcgtacagggatgggtttgtgtgtaccagatcactcacggcaaggcagacgctgacgcgctcgtcgtctccgcttcggcggggaaagactttgagctccggctacctgtggacgctagacagcaaactacgaccactggcgaatctgccgaccccgtcaccactaccgttgagaactacggaggagaaacacaaactcaacgtcgccaccacactgacgttgccttcgttcttgaccggtttgtgaaggtccaggtgtcgggcaaccaacacacactggacgttatgcaggtacacaaggacagtattgtgggtgcactcctacgcgcagccacatactacttctctgacttggaaatagcagtgactcacactgggaagctcacatgggtgcccaacggcgccccagtttctgcacttgacaacacaaccaaccccactgcctaccacaaggggccgctgactcggctggctctcccatacaccgcaccacaccgcgtgctggccacggcgtacaccggtacaacggcctacactaccggtgtacgcaggggagacctagcccacttggcggcggcgcacgctcggcacctgccgacgtcgttcaactttggtgcagttaaagcagagacaatcacagagctgcttgtgcgcatgaagcgtgctgaactctactgccccagaccggtccttccggtccaaccagcgggcgataggcacaaacaaccgctcattgcgccagcgaaacagcttctgaattttgacctgcttaagctagccggagacgttgagtccaaccctgggcccttcttcttctccgacgttaggtcaaacttttccaagctggtagacacaatcaaccagatgcaggaagacatgtccacaaagcacggacctgactttaaccggttggtgtccgcttttgaggagttggccactggagtgaaagccatcaggaccggtcttgacgaggccaagccctggtacaagcttatcaagctcctgagccgcctgtcgtgcatggccgctgtggcagcacggtcaaaggacccagtccttgtggccatcatgctggctgacaccggtctcgagattctggacagcaccttcgtcgtgaagaagatctccgactcgctctccagtctcttccacgtgccggcccccgtcttcagtttcggagccccgattctgttagccgggttggtcaaggtcgcctcgagtttcttccggtccacgcccgaagaccttgagagagcagagaaacagctcaaagcacgtgacatcaacgacattttcgccattctcaagaacggcgagtggctggtcaaattgatccttgccatccgcgactggatcaaggcatggatagcctcagaagaaaagtttgtcaccacgacagacttggtacctagcatccttgaaaaacagcaggacctcaacgacccaagcaagtacaaggaagccaaggagtggctcgacaacgcgcgccaagcgtgtttgaagagcgggaacgtccacattgccaacctgtgcaaagtggtcgccccggcacccagcaggtcgagacccgagcccgtggtcgtttgcctccgtggcaagtccggtcagggcaagagtttccttgcaaacgtgctcgcacaagcaatctctacccatttcactggcaggaccgattcagtttggtactgcccgcctgaccctgaccacttcgacggttacaaccaacagactgtcgttgtgatggacgatttgggccagaaccccgacggcaaagacttcaagtacttcgcccaaatggtttcaacaacggggttcatcccgcccatggcatcgcttgaggataaaggcaaacccttcaacagtaaggtcatcatagcaaccaccaacctgtactcgggcttcaccccgaggactatggtgtgccctgatgccctgaaccggaggtttcactttgacatcgacgtgagcgccaaggacgggtacaaaattaacaacaaattggacatcatcaaagcacttgaagatactcacaccaacccagtggcaatgtttcagtacgactgtgcccttctcaacggcatggctgttgaaatgaagagaatgcaacaagatatgttcaagcctcaaccaccccttcagaacgtgtaccaactggttcaagaggtgattgagcgggtggagctccacgagaaggtgtcgagccacccgattttcaaacagatctcaattccttcccaaaaatccgtgttgtacttcctcattgagaaaggacagcacgaggcagcaattgaattctttgagggcatggtgcacgactccatcaaggaggagctccggccgctcatccaacaaacctcatttgtgaaacgcgcttttaagcgcctgaaggaaaactttgagattgttgccctatgtctgaccctcctggccaacatagtgatcatgatccgcgaaactcgcaagagacagaagatggtggacgatgcagtgagtgagtacattgagagagcaaacatcaccaccgacgacaagactcttgatgaggcggaaaagaaccctctggaaaccagcggtgccagcaccgtcggcttcagagagagacctctcccaggccaaaaggcgcgtaatgacgagaactccgagcccgcccagcctgctgaagagcaaccacaagctgaaggaccctacgctggcccgatggagagaccagttaaagttaaagtgaaagcaaaagccccggtcgttaaggaaggaccttacgagggaccggtgaagaagcctgttgctttgaaagtgaaagctaagaacttgatcgtcactgagagtggtgccccaccgaccgacttgcaaaagttggtcatgggcaacaccaagcccgttgagctcatccttgacgggaagacggtagccatttgctgtgctactggagttttcggcactgcttacctcgtgcctcgtcatcttttcgcagaaaagtacgacaagatcatgttggacggcagagccatgacagatagtgactacagagtgtttgagtttgagattaaagtaaaaggacaggacatgctctcagacgctgcgctcatggtgctccaccgtgggaatcgcgtgagagacatcacgaaacactttcgtgacacagcaagaatgaagaaaggcacccccgtcgttggtgtgatcaacaacgccgatgtcgggagactgattttctctggtgaagcccttacctacaaggacattgtagtgtgcatggatggagacaccatgcctgggctctttgcctacaaagccgcaaccaaggctggttattgcggaggagccgtcctcgctaaggacggggctgacacgttcatcgttggcacccactccgctggaggcaatggcgttggatactgctcttgcgtttccaggtccatgcttctcaagatgaaggcacacgttgaccccgaaccacaccacgaggggttgattgttgacaccagagatgtggaagagcgcgttcacgtgatgcgcaaaaccaagcttgcacccaccgttgcgtacggtgtgttccgtcctgagttcgggcctgccgccttgtccaacaaggacccgcgcctgaacgacggtgttgtcctcgacgaagtcatcttctccaaacacaagggagacacaaagatgtctgaggaagacaaagcgctgttccgccgctgtgctgctgactacgcgtcacgcctgcacagcgtgttgggtacggcaaatgccccactgagcatctacgaggcaattaaaggcgttgatggactcgacgcaatggaaccagacaccgcacccggcctcccctgggcactccaggggaagcgccgtggcgcgctcatcgacttcgagaacggcactgttggacccgaagttgaggctgccttgaagctcatggagaaaagagaatacaagtttgcttgccaaaccttcctgaaggacgagattcgcccgatggagaaagtacgtgccggtaagactcgcattgtcgacgtcctacctgttgaacacatcctctacaccaggatgatgattggcagattttgtgcacaaatgcactcaaacaacggaccccaaattggctcggcggtcggttgtaaccctgatgttgattggcaaagatttggcacacacttcgcccaatacagaaacgtgtgggatgtggactattcggccttcgatgctaaccactgcagtgacgccatgaacatcatgtttgaggaagtgtttcgcacagaattcgggttccacccaaacgctgagtggatcctgaagactctcgtgaacacggaacacgcctatgagaacaaacgcatcactgttgaaggcgggatgccatctggttgttccgcaacaagcatcatcaacacaattttgaacaacatctacgtgctctacgctttgcgtagacactatgagggagttgagctggacacttacaccatgatctcttacggagacgatatcgtggtggcaagtgattacgatttggactttgaggctctcaagccccacttcaaatcccttggtcaaaccatcactccagctgacaaaagcgacaaaggttttgttcttggtcactccattactgatgtcactttcctcaaaagacacttccacatggattatggaactgggttttacaaacctgtgatggcctcaaagacccttgaggctatcctctcctttgcacgccgtgggaccatacaggagaagttgatctccgtggcaggactcgctgttcactctggaccagacgagtaccggcgtctcttcgagccctttcaaggcctcttcgagattccaagctacagatcactttacctgcgttgggtgaacgccgtgtgcggcgacgcataatccctcagagactacattggcatactgtttctgaggcgcgcgacgccgtaggagtgaaaagcctgaaagggcttttcccgcttcctattccaaaaaaaaaaaaaaaaaa

SEQ ID NO: 27 - DNA, 2076 bp, synthetic construct
  misc_feature 1..2076: note = Fusion Nucleotide Sequence containing capsid and 2A partial sequence of C3 Indaial strain of FMD
  source 1..2076: mol_type = other DNA
ggggccggccaatccagcccagctactggctcgcagaaccaatctggtaacacaggtagcataatcaacaactactacatgcaacagtaccaaaactccatggacacacagcttggtgacaatgccatcagtggaggctctaacgagggctccacggacacaacttcaactcacacaaccaacacccaaaacaatgactggttttcaagactcgccggttcggccttctccggtttgtttggggccttgcttgccgacaagaagacggaggagacgacactccttgaggaccgcattctcaccactcgcaatgggcacaccacctccacgacccagtccagcgtaggcgttacatacgggtactccacaacagaggaccacgttgctggacccaacacatcaggtttggagacacgagtggtacaggcagagagattctacaaaaagtttttgtttgattggacaacggacaagccttttggacacctgcacaaactggagttgcccaccgaccaccacggtgttttcggacacttggtggactcatacgcctacatgaggaacggttgggacgttgaggtgtctgctgttggcaaccagttcaacggcggatgcctcctagtggccatggtacccgaatggaaagagtttgaaacgcgggagaagtaccagctcacgcttttcccgcaccagttcattagccccagaaccaacatgaccgcccacatcacggttccttaccttggtgtgaatagatatgatcagtacaaaaaacacaaaccctggacactggttgtcatggtcgtgtccccgctcacggtcaacgccacgagcgcggcacagatcaaggtctatgccaacatcgctccgacctacgttcatgtggccggcgagctcccctcgaaagaggggatcttccctgtcgcgtgcgcggacggttacggaggactggtgacaacggacccgaaaacagctgaccccgcctacggcaaggtgtacaatccgccccggactaactaccccgggcgtttcactaacttgttggacgtggctgaggcatgtcccacctttctgtgttttgacgacgggaaaccgtacgttaccacacagacaggtgagtctcgtcttctggccaagttcgacctttcccttgccgcgaagcacatgtctaacacatacttggcaggaattgcccagtactacacacagtactcaggcaccatcaatttgcatttcatgttcacaggttcaactgattcaaaagcccgctacatggtggcttacatcccgcctggggtggaaacaccaccggacacacctgagagggcagcccactgcatccatgctgagtgggacacagggctgaattccaaattcacattctcaatcccgtacgtgtctgccgcggattacgcctacacggcgtctgatgaggcagagacaacaaacgtacagggatgggtctgcgtttaccagatcacacacgggaaggctgacaacgacactctggtcgtgtcggttagcgccggcaaggacttcgagttgcgcctccccattgacccccgaccgcagaccaccgctactggggaatcagcagaccctgtcaccaccactgtagagaactacggcggtgagacacaagttcagagacgccaccacaccgacgttggcttcatcatggacagatttgtgaaaataaacagcccaaaatccacccatgttattgacctcatgcaaacccaccaacacggtctagtgggtgcgctgctgcgtgcggcgacctactacttctcagatctggaaattgttgtgcggcatgacggcaacctaacttgggtgcccaatggtgctcccgtgtcagccttgtccaacaccagcaaccccaccgcctacaacaaggcaccgttcacgagacttgccctcccctacaccgcgccacaccgcgtgttggcgactgtgtacaacgggacgagcaagtacactgtgagtgggtcaagcagacgaggcgacttgggttccctcgcggcacgagtcgtgaaggcacttcctgcttctttcaactacggtgcaatcaag

SEQ ID NO: 28 - DNA, 23 bp, synthetic construct
  misc_feature 1..23: note = Primer
  source 1..23: mol_type = other DNA
gacaaaggttttgttcttggtca

SEQ ID NO: 29 - DNA, 18 bp, synthetic construct
  misc_feature 1..18: note = Primer
  source 1..18: mol_type = other DNA
tgcgagtcctgccacgga

SEQ ID NO: 30 - DNA, 20 bp, synthetic construct
  misc_feature 1..20: note = Probe
  source 1..20: mol_type = other DNA
tcctttgcacgccgtgggac

(19) United States
(12) Patent Application Publication
(10) Pub. No.: US 2024/0197855 A1
(43) Pub. Date: Jun. 20, 2024

(54) FOOT-AND-MOUTH DISEASE VACCINE

(71) Applicants: Zoetis Services LLC, Parsippany, NJ (US); United States of America, as Represented by the Secretary of Agriculture, Washington, DC (US)

(72) Inventors: Paul Joseph Dominowski, Kalamazoo, MI (US); John Morgan Hardham, Kalamazoo, MI (US); James Alan Jackson, Kalamazoo, MI (US); Cyril Gerard Gay, Bethesda, MD (US); Luis Leandro Rodriguez, Niantic, CT (US); Peter William Krug, East Setauket, NY (US); Aida Elizabeth Rieder, Westbrook, CT (US)

(21) Appl. No.: 18/511,123

(22) Filed: Nov. 16, 2023

Related U.S. Application Data
(63) Continuation of application No. 17/180,223, filed on Feb. 19, 2021, now Pat. No. 11,865,171.
Application No. 17/180,223 is a continuation of application No. 16/374,105, filed on Apr. 3, 2019, now Pat. No. 10,967,058.
Application No. 16/374,105 is a continuation of application No. 15/543,630, filed on Jul. 14, 2017, now Pat. No. 10,478,487.
(60) Provisional application No. 62/104,314, filed on Jan. 16, 2015.

(51) Int. Cl. A61K 39/135; A61K 39/00; A61K 39/12; A61K 39/39

(52) U.S. Cl. CPC A61K 39/135; A61K 39/12; A61K 39/39; A61K 2039/5252; A61K 2039/552; A61K 2039/555; A61K 2039/55505; A61K 2039/55511; A61K 2039/55561; A61K 2039/55566; A61K 2039/55583; C12N 2770/32034; C12N 2770/32134

(57) ABSTRACT

Compositions for prevention of Foot and Mouth Disease (FMD) are provided, comprising an antigen component in the amount equivalent to 0.5-20 μg FMD virus and an adjuvant component comprising oil, an immunostimulatory oligonucleotide, and a polycationic carrier. Methods of using the composition, as well as the methods of reducing FMD persistence are also provided.

### CROSS REFERENCE TO RELATED APPLICATIONS

This application is a continuation of U.S. application Ser. No. 17/180,223 filed on Feb. 19, 2021, issued as U.S. Pat. No. 11,865,171, which is a continuation of U.S. application Ser. No. 16/374,105 filed on Apr. 3, 2019, issued as U.S. Pat. No. 10,967,058, which is a continuation of U.S. application Ser. No. 15/543,630 filed on Jul. 14, 2017, issued as U.S. Pat. No. 10,478,487, which is the National Stage of International Application No. PCT/US2016/013587, filed on Jan. 15, 2016, which application claims the benefit of U.S. Provisional Application No. 62/104,314, filed Jan. 16, 2015, now expired.

### THE NAMES OF THE PARTIES TO A JOINT RESEARCH AGREEMENT

The claimed invention was made as a result of activities undertaken within the scope of a joint research agreement between Zoetis LLC and the United States Department of Agriculture, Agricultural Research Service.

### SEQUENCE LISTING INFORMATION

The instant application contains a Sequence Listing which has been submitted electronically in XML file format and is hereby incorporated by reference in its entirety. Said XML copy, created on Nov. 15, 2023, is named ZP000077D.XML and is 95,757 bytes in size.

### BACKGROUND

Foot and mouth disease (FMD) is an extremely contagious viral disease of cloven-hoofed ungulates which include domestic animals (cattle, pigs, sheep, goats, and others) and a variety of wild animals. The most prominent disease symptoms in FMDV-infected cattle include vesicular lesions of the epithelium of the mouth, tongue, teats and feet. Although some countries, among them United States, Canada, Mexico, Australia and most of Europe, are considered to be free of FMD, the disease is distributed worldwide and has a great economic impact on the export industry. Indeed, several economically devastating outbreaks have occurred over the past decade on almost every continent.

Currently killed-antigen FMDV vaccines are necessarily produced in expensive biological containment facilities, by growing large volumes (thousands of liters) of virulent FMDV that has been adapted to grow in cells, which can be sometimes difficult. This process has resulted in escape of virulent virus from the manufacturing facility causing costly outbreaks in livestock (see Cottam et al. 2008. PLOS Pathogen 4:1-8). After growth, virus is then inactivated using chemicals and antigen concentrates are prepared, followed by purification steps required to remove contaminant proteins. It is difficult to differentiate infected from vaccinated animals (DIVA) through serological diagnostic tests. There is little to no cross protection across serotypes and subtypes requiring the appropriate matching between vaccine and circulating field strains to achieve protection. Despite these shortcomings of the vaccines, billions of doses are manufactured every year around the world. Their use has been the basis for eradicating FMDV from Europe and for controlling the disease in many parts of the world through mass vaccination campaigns. Creation of genetically engineered viruses containing a backbone and suitable restriction sites partially addresses the shortcomings of inactivated vaccines as restriction sites provide loci for introduction of capsid proteins of different FMD strains. Nevertheless, the cost of antigen is the greatest contributor to the cost of FMD and most other vaccines.

The problem of FMD control is further exacerbated by the phenomenon of virus persistence. Briefly, historically, inactivated FMD vaccines have been unable to prevent persistence or carrier state (defined as virus shedding past 28 days following infection and/or exposure). Shedding animals, while not exhibiting any FMD symptoms, could remain a source of FMD infection to other animals. As such, commonly accepted disease control practices require slaughter of all animals in a vaccinated herd even if they do not have clinical signs of disease.

As such, methods and compositions which lead to vaccines with a lower antigen load without compromising efficiency and/or reducing or eliminating FMD persistence are still desired.

### SUMMARY OF THE INVENTION

In one aspect, the invention provides an immunogenic composition comprising an antigen component and an adjuvant component, wherein the adjuvant component comprises an emulsion containing an oily phase, said oily phase comprising at least 50% v/v of said immunogenic composition, an immunostimulatory oligonucleotide, and at least one of a polycationic polymer; a source of aluminum; and the antigen component comprises a FMD antigen composition in the amount equivalent to 0.5-8 μg of FMD virus per dose.

In certain embodiments, the immunostimulatory oligonucleotide is a CpG containing oligonucleotide. In certain embodiments, the polycationic polymer is DEAE dextran.

In different embodiments, the antigen is an FMD virus composition, and is present in the amount of 0.5-4 μg per dose, or 0.5-2 μg per dose, or 0.5-1 μg per dose, or in the amount of about 0.5 μg per dose.

The FMD virus may be inactivated or attenuated. In certain embodiments, the FMD virus is an inactivated FMD A24 Cruzeiro strain. In selected embodiments, the inactivated strain is a genetically engineered strain which contains a deletion of the leader coding region (LL) and optionally, contains negative antigenic markers.

In certain embodiments, the genetically engineered virus contains capsid proteins from a heterologous strain.

In another aspect, the invention provides a method of preventing FMD in an animal in need thereof, the method comprising administering the immunogenic composition according to the embodiments of the previous aspect to said animal. In different embodiments, the animal is selected from bovines, ovines, porcines, and caprines.

In another aspect, the invention provides a method of reducing frequency of FMD persistence in a ruminant infected with FMD comprising administering to said ruminant prior to the infection an immunogenic composition comprising an antigen component and an adjuvant component, wherein the adjuvant component comprises an emulsion containing an oily phase, said oily phase comprising at least 50% v/v of said immunogenic composition, an immunostimulatory oligonucleotide in the amount of 75-200 μg per dose, and a polycationic polymer in the amount of 75-200 mg per dose; and the antigen component comprises a FMD antigen in the amount equivalent to 6-10 μg of FMD virus per dose.

In yet another aspect, the invention provides a method of herd management, comprising administering to animals in said herd an immunogenic composition comprising an antigen component and an adjuvant component, wherein the adjuvant component comprises an emulsion containing an oily phase, said oily phase comprising at least 50% v/v of said immunogenic composition, an immunostimulatory oligonucleotide in the amount of 75-200 μg per dose, and a polycationic polymer in the amount of 75-200 mg per dose; and the antigen component comprises a FMD antigen in the amount equivalent to 6-10 μg of FMD virus per dose, wherein, upon suspected contact with FMD infection, the vaccinated members of the herd are not slaughtered.

The invention also provides a method of herd management, comprising administering to animals in said herd an immunogenic composition comprising an antigen component and an adjuvant component, wherein the adjuvant component comprises an emulsion containing an oily phase, said oily phase comprising at least 50% v/v of said immunogenic composition, an immunostimulatory oligonucleotide in the amount of 75-200 μg per dose, and a polycationic polymer in the amount of 75-200 mg per dose; and the antigen component comprises a FMD antigen in the amount equivalent to 6-10 μg of FMD virus per dose, wherein, upon suspected contact with FMD infection, the vaccinated members of the herd are quarantined for 0-62 days.

The invention also provides a method of herd management, comprising administering to animals in said herd an immunogenic composition comprising an antigen component and an adjuvant component, wherein the adjuvant component comprises an emulsion containing an oily phase, said oily phase comprising at least 50% v/v of said immunogenic composition, an immunostimulatory oligonucleotide in the amount of 75-200 μg per dose, and a polycationic polymer in the amount of 75-200 mg per dose; and the antigen component comprises a FMD antigen in the amount equivalent to 6-10 μg of FMD virus per dose, wherein, upon suspected contact with FMD infection, the vaccinated members of the herd are moved beyond the infected zone.

### BRIEF DESCRIPTION OF THE DRAWINGS

FIG. 1 illustrates the difference in quality between the PEG precipitated and hollow fiber concentrated antigens.

### DETAILED DESCRIPTION

### Definitions

“About” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater, unless about is used in reference to time intervals in weeks where “about 3 weeks,” is 17 to 25 days, and about 2 to about 4 weeks is 10 to 40 days.

“Adjuvant” means any substance that increases the humoral or cellular immune response to an antigen. Adjuvants are generally used to accomplish two objectives: the controlled release of antigens from the injection site, and the stimulation of the immune system.

“Antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions.

“Antigen” or “immunogen” refers to any substance that is recognized by the animal's immune system and generates an immune response. The term includes killed, inactivated, attenuated, or modified live bacteria, viruses, or parasites. The term “antigen” also includes polynucleotides, polypeptides, recombinant proteins, synthetic peptides, protein extract, cells (including tumor cells), tissues, polysaccharides, or lipids, or fragments thereof, individually or in any combination thereof. The term antigen also includes antibodies, such as anti-idiotype antibodies or fragments thereof, and to synthetic peptide mimotopes that can mimic an antigen or antigenic determinant (epitope).

“Buffer” means a chemical system that prevents change in the concentration of another chemical substance, e.g., proton donor and acceptor systems serve as buffers preventing marked changes in hydrogen ion concentration (pH). A further example of a buffer is a solution containing a mixture of a weak acid and its salt (conjugate base) or a weak base and its salt (conjugate acid).

“Consisting essentially” as applied to the adjuvant formulations refers to formulation which does not contain unrecited additional adjuvanting or immunomodulating agents in the amounts at which said agent exert measurable adjuvanting or immunomodulating effects.

“Dose” refers to a vaccine or immunogenic composition given to a subject. A “first dose” or “priming vaccine” refers to the dose of such a composition given on Day 0. A “second dose” or a “third dose” or an “annual dose” refers to an amount of such composition given subsequent to the first dose, which may or may not be the same vaccine or immunogenic composition as the first dose.

The term “emulsifier” is used broadly in the instant disclosure. It includes substances generally accepted as emulsifiers, e.g., different products of TWEEN® or SPAN® product lines (fatty acid esters of polyethoxylated sorbitol and fatty-acid-substituted sorbitan surfactants, respectively), and different solubility enhancers such as PEG-40 Castor Oil or another PEGylated hydrogenated oil.

“Humoral immune response” refers to one that is mediated by antibodies.

“Immune response” in a subject refers to the development of a humoral immune response, a cellular immune response, or a humoral and a cellular immune response to an antigen. Immune responses can usually be determined using standard immunoassays and neutralization assays, which are known in the art.

“Immunologically effective amount” or “effective amount to produce an immune response” of an antigen is an amount effective to induce an immunogenic response in the recipient. The immunogenic response may be sufficient for diagnostic purposes or other testing, or may be adequate to prevent signs or symptoms of disease, including adverse health effects or complications thereof, caused by infection with a disease agent. Either humoral immunity or cell-mediated immunity or both may be induced. The immunogenic response of an animal to an immunogenic composition may be evaluated, e.g., indirectly through measurement of antibody titers, lymphocyte proliferation assays, or directly through monitoring signs and symptoms after challenge with wild type strain, whereas the protective immunity conferred by a vaccine can be evaluated by measuring, e.g., reduction in clinical signs such as mortality, morbidity, temperature number, overall physical condition, and overall health and performance of the subject. The immune response may comprise, without limitation, induction of cellular and/or humoral immunity.

“Immunogenic” means evoking an immune or antigenic response. Thus an immunogenic composition would be any composition that induces an immune response.

“Infected Premises” refers to premises where presumptive positive case or confirmed positive case exists based on laboratory results, compatible clinical signs, FMD case definition, and international standards.

“Infected Zone” refers to an area within 3 km beyond perimeters of presumptive or confirmed Infected Premises.

“Lipids” refers to any of a group of organic compounds, including the fats, oils, waxes, sterols, and triglycerides that are insoluble in water but soluble in nonpolar organic solvents, are oily to the touch, and together with carbohydrates and proteins constitute the principal structural material of living cells.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

“TCID₅₀” refers to “tissue culture infective dose” and is defined as that dilution of a virus required to infect 50% of a given batch of inoculated cell cultures. Various methods may be used to calculate TCID₅₀, including the Spearman-Karber method which is utilized throughout this specification. For a description of the Spearman-Karber method, see B. W. Mahy & H. O. Kangro, Virology Methods Manual, p. 25-46 (1996).

Persistently infected or carrier animals are animals shedding FMD virus past 28 days post infection or onset of clinical disease.

### Adjuvant Formulations and Methods of Making

The instant application discloses several adjuvant formulations suitable for the instant invention. The common feature of these adjuvants is the presence of oil and one or more emulsifiers, wherein the oily phase comprises at least 50% of the vaccine composition encompassing the adjuvant formulations disclosed therein.

Multiple oils and combinations thereof are suitable for use of the instant invention. These oils include, without limitations, animal oils, vegetable oils, as well as non-metabolizable oils. Non-limiting examples of vegetable oils suitable in the instant invention are corn oil, peanut oil, soybean oil, coconut oil, and olive oil. A non-limiting example of an animal oil is squalane. Suitable non-limiting examples of non-metabolizable oils include light mineral oil, straight chained or branched saturated oils, and the like.

In a set of embodiments, the oil used in the adjuvant formulations of the instant invention is a light mineral oil. As used herein, the term “mineral oil” refers to a mixture of liquid hydrocarbons obtained from petrolatum via a distillation technique. The term is synonymous with “liquefied paraffin”, “liquid petrolatum” and “white mineral oil.” The term is also intended to include “light mineral oil,” i.e., oil which is similarly obtained by distillation of petrolatum, but which has a slightly lower specific gravity than white mineral oil. See, e.g., Remington's Pharmaceutical Sciences, 18th Edition (Easton, Pa.: Mack Publishing Company, 1990, at pages 788 and 1323). Mineral oil can be obtained from various commercial sources, for example, J. T. Baker (Phillipsburg, Pa.) or USB Corporation (Cleveland, Ohio). Preferred mineral oil is light mineral oil commercially available under the name DRAKEOL®.

In certain embodiments particularly suitable for preventing or eliminating FMD persistence, the oily phase is present in an amount from 50% to 95% by volume; preferably, in an amount of greater than 50% to 85%; more preferably, in an amount from greater than 50% to 60%, and more preferably in the amount of greater than 50-52% v/v of the vaccine composition. The oily phase includes oil and emulsifiers (e.g., SPAN® 80, TWEEN® 80, etc.), if any such emulsifiers are present. The volume of the oily phase is calculated as a sum of volumes of the oil and the emulsifier(s). Thus, for example, if the volume of the oil is 40% and the volume of the emulsifier(s) is 12% of a composition, then the oily phase would be present at 52% v/v of the composition. Similarly, if the oil is present in the amount of about 45% and the emulsifier(s) is present in the amount of about 6% of a composition, then the oily phase is present at about 51% v/v of the composition.

It also should be understood that since the adjuvants of the instant invention form only a part of the vaccines of the instant invention, the oily phase is present in an amount from 50% to 95% by volume; preferably, in an amount of greater than 50% to 85%; more preferably, in an amount from 50% to 60%, and more preferably in the amount of 50-52% v/v of each of the adjuvants of the instant invention.

In a subset of embodiments, the volume percentage of the oil and the oil-soluble emulsifier together is at least 50%, e.g., 50% to 95% by volume; preferably, in an amount of greater than 50% to 85%; more preferably, in an amount from 50% to 60%, and more preferably in the amount of 50-52% v/v of the vaccine composition. Thus, for example and without limitations, the oil may be present in the amount of 45% and the lipid-soluble emulsifier would be present in the amount of greater than 5% v/v. Thus, the volume percentage of the oil and the oil-soluble emulsifier together would be at least 50%.

In yet another subset, applicable to all vaccines of the invention, volume percentage of the oil is over 40%, e.g., 40% to 90% by volume; 40% to 85%; 43% to 60%, 44-50% v/v of the vaccine composition.

Emulsifiers suitable for use in the present emulsions include natural biologically compatible emulsifiers and non-natural synthetic surfactants. Biologically compatible emulsifiers include phospholipid compounds or a mixture of phospholipids. Preferred phospholipids are phosphatidylcholines (lecithin), such as soy or egg lecithin. Lecithin can be obtained as a mixture of phosphatides and triglycerides by water-washing crude vegetable oils, and separating and drying the resulting hydrated gums. A refined product can be obtained by fractionating the mixture for acetone insoluble phospholipids and glycolipids remaining after removal of the triglycerides and vegetable oil by acetone washing. Alternatively, lecithin can be obtained from various commercial sources. Other suitable phospholipids include phosphatidylglycerol, phosphatidylinositol, phosphatidylserine, phosphatidic acid, cardiolipin, and phosphatidylethanolamine. The phospholipids may be isolated from natural sources or conventionally synthesized.

In additional embodiments, the emulsifiers used herein do not include lecithin, or use lecithin in an amount which is not immunologically effective.

Non-natural, synthetic emulsifiers suitable for use in the adjuvant formulations of the present invention include sorbitan-based non-ionic surfactants, e.g. fatty-acid-substituted sorbitan surfactants (commercially available under the name SPAN® or ARLACEL®), fatty acid esters of polyethoxylated sorbitol (TWEEN®), polyethylene glycol esters of fatty acids from sources such as castor oil (EMULFOR®); polyethoxylated fatty acid (e.g., stearic acid available under the name SIMULSOL® M-53), polyethoxylated isooctylphenol/formaldehyde polymer (TYLOXAPOL®), polyoxyethylene fatty alcohol ethers (BRIJ®); polyoxyethylene nonphenyl ethers (TRITON® N), polyoxyethylene isooctylphenyl ethers (TRITON® X). Preferred synthetic surfactants are the surfactants available under the name SPAN® and TWEEN®, such as TWEEN®-80 (Polyoxyethylene (20) sorbitan monooleate) and SPAN®-80 (sorbitan monooleate).

Generally speaking, the emulsifier(s) may be present in the vaccine composition in an amount of 0.01% to 40% by volume, preferably, 0.1% to 15%, more preferably 2% to 10%. Additional ingredients present in the instant adjuvant formulations include cationic carriers, immunostimulatory oligonucleotides, monophospholipid A and analogs thereof (MPL-A), Polyinosinic:polycytidylic acid (poly:C), saponins, quaternary ammoniums, sterols, glycolipids, a source of aluminum (e.g., REHYDRAGEL® or VAC 20® wet gel) and combinations thereof.

Suitable cationic carriers include, without limitations, dextran, dextran DEAE (and derivatives thereof), PEGs, guar gums, chitosan derivatives, polycellulose derivatives like hydroxyethyl cellulose (HEC) polyethylenimene, poly aminos like polylysine and the like.

Suitable immunostimulatory oligonucleotides include ODN (DNA-based), ORN (RNA-based) oligonucleotides, or chimeric ODN-ORN structures, which may have modified backbone including, without limitations, phosphorothioate modifications, halogenations, alkylation (e.g., ethyl- or methyl-modifications), and phosphodiester modifications. In some embodiments, poly inosinic-cytidylic acid or derivative thereof (poly I:C) may be used.

CpG oligonucleotides are a recently described class of pharmacotherapeutic agents that are characterized by the presence of an unmethylated CG dinucleotide in specific base-sequence contexts (CpG motif). (Hansel T T, Barnes P J (eds): New Drugs for Asthma, Allergy and COPD. Prog Respir Res. Basel, Karger, 2001, vol 31, pp 229-232, which is incorporated herein by reference). These CpG motifs are not seen in eukaryotic DNA, in which CG dinucleotides are suppressed and, when present, usually methylated, but are present in bacterial DNA to which they confer immunostimulatory properties.

In selected embodiments, the adjuvants of the instant invention utilize a so-called P-class immunostimulatory oligonucleotide, more preferably, modified P-class immunostimulatory oligonucleotides, even more preferably, E-modified P-class oligonucleotides. P-class immunostimulatory oligonucleotides are CpG oligonucleotides characterized by the presence of palindromes, generally 6-20 nucleotides long. The P-Class oligonucleotides have the ability to spontaneously self-assemble into concatamers either in vitro and/or in vivo. These oligonucleotides are, in a strict sense, single-stranded, but the presence of palindromes allows for formation of concatamers or possibly stem-and-loop structures. The overall length of P-class immunostimulatory oligonucleotides is between 19 and 100 nucleotides, e.g., 19-30 nucleotides, 30-40 nucleotides, 40-50 nucleotides, 50-60 nucleotides, 60-70 nucleotides, 70-80 nucleotides, 80-90 nucleotides, 90-100 nucleotides.

In one aspect of the invention the immunostimulatory oligonucleotide contains a 5′ TLR activation domain and at least two palindromic regions, one palindromic region being a 5′ palindromic region of at least 6 nucleotides in length and connected to a 3′ palindromic region of at least 8 nucleotides in length either directly or through a spacer.

The P-class immunostimulatory oligonucleotides may be modified according to techniques known in the art. For example, J-modification refers to iodo-modified nucleotides. E-modification refers to ethyl-modified nucleotide(s). Thus, E-modified P-class immunostimulatory oligonucleotides are P-class immunostimulatory oligonucleotides, wherein at least one nucleotide (preferably 5′ nucleotide) is ethylated. Additional modifications include attachment of 6-nitro-benzimidazol, O-methylation, modification with proynyl-dU, inosine modification, 2-bromovinyl attachment (preferably to uridine).

The P-class immunostimulatory oligonucleotides may also contain a modified internucleotide linkage including, without limitations, phosphodiesther linkages and phosphorothioate linkages. The oligonucleotides of the instant invention may be synthesized or obtained from commercial sources.

P-Class oligonucleotides and modified P-class oligonucleotides are further disclosed in published PCT application no. WO2008/068638, published on Jun. 12, 2008. Suitable non-limiting examples of modified P-class immunostimulatory oligonucleotides are provided below (“*” refers to a phosphorothioate bond and “-” refers to a phosphodiester bond).

SEQ ID NO: 1
5′ T*C-G*T*C-G*A*C-G*A*T*C-G*G*C*G*C-G*C*G*C*C*G
3′
SEQ ID NO: 2
5′ T*C-G*A*C*G*T*C*G*A*T*C*G*G*C*G*C*G*C*G*C*C*G
3′
SEQ ID NO: 3
5′ T*C*G*A*C*G*T*C*G*A*T*C*G*G*C*G*C*G*C*G*C*C*G*
T 3′
SEQ ID NO: 4
5′ JU*C-G*A*C*G*T*C*G*A*T*C*G*G*C*G*C*G*C*G*C*C*G
3′
SEQ ID NO: 5
5′ JU*C-G*A*C*G*T*C*G*A*T*C*G*G*C*G*C*G*C*G*C*C*
G*T 3′
SEQ ID NO: 6
5′ JU*C*G*A*C*G*T*C*G*A*T*C*G*G*C*G*C*G*C*G*C*C*
G*T 3′
SEQ ID NO: 7
5′ EU*C-G*A*C*G*T*C*G*A*T*C*G*G*C*G*C*G*C*G*C*C*G
3′
SEQ ID NO: 8
5′ JU*C-G*T*C*G*A*C*G*A*T*C*G*G*C*G*G*C*C*G*C*C*
G*T 3′
SEQ ID NO: 9
5′ JU*C*G*T*C*G*A*C*G*A*T*C*G*G*C*G*G*C*C*G*C*C*
G*T 3′
SEQ ID NO: 10
5′ T*C-G*T*C-G*A*C-G*A*T*C-G*G*C*G*C-G*C*G*C*C*G
3′
SEQ ID NO: 11
5′-UUGUUGUUGUUGUUGUUGUU-3′
SEQ ID NO: 12
5′-UUAUUAUUAUUAUUAUUAUU-3′
SEQ ID NO: 13
5′-AAACGCUCAGCCAAAGCAG-3′
SEQ ID NO: 14
dTdCdGdTdCdGdTdTdTdTrGrUrUrGrUrGrUdTdTdTdT-3′

The amount of P-class immunostimulatory oligonucleotide for use in the adjuvant compositions depends upon the nature of the P-class immunostimulatory oligonucleotide used and the intended species.

In addition to the oil and the emulsifier(s), the adjuvant formulations also comprise (or consist essentially of, or consist of) a combination of an immunostimulatory oligonucleotide and a polycationic carrier. These adjuvants are referred to as “TXO”.

In a set of embodiments, the TXO adjuvants may also include a source of aluminum, such as Al(OH)₃ gel. The TXO adjuvants with aluminum are referred to as “TXO-A”.

In a set of embodiments, adjuvants TXO and TXO-A may optionally contain a sterol, such as, for example, cholesterol, lanosterol, sigmasterol, etc. TXO and TXO-A adjuvants containing the sterol are referred to as TCXO and TCXO-A, respectively. The optionally present sterol may be present in the amount of up to about 1000 μg (e.g., 100-1000 μg, 200-1000 μg, 250-700 μg, or about 400-500 μg) per dose.

In a set of embodiments, in TXO adjuvants, the immunostimulatory oligonucleotide, preferably an ODN, preferably containing a palindromic sequence, and optionally with a modified backbone, may be present in the amount of 5-400 μg per dose, and the polycationic carrier may be present in the amount of 5-400 mg per dose.

For example, in certain embodiments, one dose of TXO would comprise between about 5 and 400 μg per dose (e.g., 6.25-200 μg or 6.25-100 μg or 6.25-50 μg or 6.25-25 μg or 6.25-10 μg or 10-200 μg or 25-200 μg or 25-100 μg or 25-50 μg or 25-100 μg or 50-100 μg per dose) of the immunostimulatory oligonucleotide, and the polycationic carrier may be present in the amount of between about 5 and about 500 mg per dose (e.g., 6.25-200 mg or 6.25-100 mg or 6.25-50 mg or 6.25-25 mg or 6.25-10 mg or 10-200 mg or 25-200 mg or 25-100 mg or 25-50 mg or 25-100 mg or 50-100 mg per dose).

In certain embodiments, TXO adjuvants are prepared as follows:
a) Sorbitan monooleate is dissolved in light mineral oil. The resulting oil solution is sterile filtered;
b) The immunostimulatory oligonucleotide, Dextran DEAE and Polyoxyethylene (20) sorbitan monooleate are dissolved in aqueous phase, thus forming the aqueous solution; and
c) The aqueous solution is added to the oil solution under continuous homogenization thus forming the adjuvant formulation TXO.

In a set of embodiments, in TXO-A adjuvants, the immunostimulatory oligonucleotide is present as in the TXO adjuvant, the source of aluminum is present in the amount of up to 40% v/v (e.g., 35%, 30%, 25%, 20%, 15%, 10%, 5%, 1%). In a set of embodiments, the source of aluminum is present at 2%-20% v/v of the vaccine composition, more preferably between about 5% and about 17% v/v.

In certain embodiments, TXO-A adjuvants are prepared similarly to TXO adjuvants, and the source of aluminum is added to the aqueous solution.

In preparation of TCXO and TCXO-A adjuvants, cholesterol is dissolved in the oil solution, and the other steps of making TCXO and TCXO-A are similar to the steps used in preparation of TXO and TXO-A, respectively.

### Antigens

The inventors have surprisingly discovered that the adjuvants of the instant invention are capable of causing sufficient protection from Foot-And-Mouth disease even when the dose of the antigen is decreased from 10 μg of the FMD virus to 0.5 μg. Thus, in different embodiments of the invention, the amount of the FMD virus may be 0.5 μg, about 1 μg, about 2 μg, about 3 μg, about 4 μg, about 5 μg, about 6 μg, about 7 μg, about 8 μg, about 9 μg or about 10 μg. The amount of the antigen may be between 0.5 and 1 μg, between 1 and 2 μg, between 2 and 3 μg, between 3 and 4 μg, between 4 and 5 μg, between 5 and 6 μg, between 6 and 8 μg, between 8 and 10 μg of FMD virus (140 S particles).

Currently, seven serotypes of FMD have been isolated. Of the seven serotypes of this virus, A, C, O, Asia 1, and SAT3 appear to be distinct lineages; SAT 1 and SAT 2 are unresolved clades. Within each serovar, multiple strains exist. For example, A24 Cruzeiro belongs to serotype A, and O1 Campos belongs to serotype O.

FMD virus of any serotype may be used as an antigen in this invention, provided that such virus is not pathogenic. Pathogenicity may be reduced by inactivation of the virus, e.g., treatment with formaldehyde or BEI.

In certain embodiments, the virus may be attenuated by culture passage or via recombinant means. It has previously been demonstrated, for example, that deletion of the leader protein L^{pro} coding region results in FMD virus which is attenuated in cattle and pigs. See, e.g., U.S. Pat. Nos. 5,824,316, 8,765,141, Virology 1997 227(1): 96-102, J. Virol 2012 86:11675-11685. Point mutations in at positions 55 and 58 within the SAP domain of L protein also resulted in a viable virus that displayed a mild attenuated phenotype in cell culture and was protective in swine FMD model. See U.S. Pat. No. 8,846,057.

In certain embodiments, the virus also contains negative antigenic markers which allow for DIVA (differentiating infected from vaccinated animals) assays. In certain embodiments, the negative antigenic markers are introduced to 3D and/or 3B proteins. See, e.g., SEQ ID NOs 19, 20, 21, 22.

Like other viruses, the FMD virus continually evolves and mutates, thus one of the difficulties in vaccinating against it is the huge variation between, and even within, serotypes. There is no cross-protection between serotypes (a vaccine for one serotype will not necessarily protect against any others) and in addition, two strains within a given serotype may have nucleotide sequences that differ by as much as 30% for a given gene. This means FMD vaccines must be highly specific to the strain involved.

Thus, in certain embodiments, endonuclease restriction sites are introduced into the genome of the virus, thereby allowing introduction of proteins (e.g., proteins forming the outer capsids) from heterologous FMD strains.

In certain embodiments, the antigen component comprises FMD strain A24 Cruzeiro, which may optionally be modified by deletion of leader protein, negative marker mutations in 3B and/or 3D proteins, and by introduction of restriction endonuclease sites for an easier introduction of sequences for antigens (e.g., capsid proteins) from heterologous strains. Suitable non-limiting examples of the antigens are described in U.S. Pat. No. 8,765,141. DNA sequences corresponding to RNA genome of a genetically modified FMDV are also provided in SEQ ID NO: 15 (A₂₄LL3D_{YR}) and SEQ ID NO: 17 (A₂₄LL3B_{PVKV}3D_{YR}). Thus, a DNA sequence complementary to the DNA sequence set forth e.g., in SEQ ID NO: 15 is a template for, i.e. is complementary to or “encodes”, the RNA genome of the FMDV virus (i.e., RNA that encodes the FMDV). In certain embodiments, the virus comprises capsid protein(s) of heterologous FMD strains (i.e., strains of FMD other than A₂₄ Cruzeiro, including without limitations, strains of lineages C, O, Asia 1, SAT3, SAT 1 and SAT 2, Turkey 06 and other strains of lineage A). Non limiting examples of such heterologous antigens are illustrated in SEQ ID NO: 23 (Asia1-A₂₄LL3B_{PVKV}3D_{YR}) and SEQ ID NO: 24 (A/Turkey/06-A₂₄LL3B_{PVKV}3D_{YR}). Additionally, O1 campos-A₂₄LL3B_{PVKV}3D_{YR} (complete genome, also referred as O1campos), C3 Indaial-A₂₄LL3B_{PVKV}3D_{YR} (complete genome), and capsid Argentina 2001 iso93 (capsid and 2A partial sequence) are provided in SEQ ID NOs 25, 26, and 27, respectively.

Variants of such antigens are also envisioned. The variants are at least 80% identical (e.g., 85% identical, 90% identical, 95% identical, 96% identical, 97% identical, 98% identical or 99% identical) to a reference sequence using one of the alignment programs described using standard parameters. Multiple alignment tools are available to determine sequence identity, including, without limitations, BLAST, CLUSTAL or PHILIP.

One of skill in the art will recognize that these values can be appropriately adjusted to determine corresponding identity of proteins encoded by two nucleotide sequences by taking into account codon degeneracy, amino acid similarity, reading frame positioning, and the like.

In certain embodiments, the variants encompass more than the specific exemplary nucleotide or amino acid sequences and include functional equivalents thereof. Alterations in a nucleic acid fragment that result in the production of a chemically equivalent amino acid at a given site, but do not affect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The polypeptides of the invention may also be altered in various ways including amino acid substitutions, deletions, truncations, and insertions. Novel proteins having properties of interest may be created by combining elements and fragments of proteins of the present invention, as well as with other proteins. Methods for such manipulations are generally known in the art. Thus, the genes and nucleotide sequences of the invention include both the naturally occurring sequences as well as mutant forms. Likewise, the proteins of the invention encompass naturally occurring proteins as well as variations and modified forms thereof. Such variants will continue to possess the desired modified activities of the parent FMD virus. The mutations that will be made in the DNA encoding the variant must not place the sequence out of reading frame and preferably will not create complementary regions that could produce secondary mRNA structure.

Methods of growing and purifying the antigens suitable for the instant invention are well known in the art and include, without limitations, hollow fiber filtration and PEG precipitation. These methods yield somewhat different antigenic compositions. For example, in PEG precipitation, the antigenic composition is depleted of non-structural proteins. In other methods, such as, for example, hollow fiber filtration, the antigenic composition contains both structural and non-structural FMD proteins. Accordingly, in some embodiments, the FMD antigen comprises structural proteins. In other embodiments, such as, for example, where the FMD antigen is prepared by hollow fiber filtration, the FMD antigen comprises both structural and non-structural proteins, particularly 3D protein.

Using current vaccine platforms, devoid of intrinsic antigenic markers to differentiate vaccinated from infected animals, removal of non-structural proteins is desirable as this remains desirable due to the fact that presence of antibodies to non-structural protein identifies infected animals. However in the context of the FMDLL3B3D platform, the presence of non-structural protein in the antigen preparation does not preclude differentiation between vaccinated and infected animals. It is in this context that the present formulation of antigen including non-structural proteins and adjuvant provide both protection against clinical disease at lower doses than purified antigen formulations and also prevent more effectively the establishment of persistent infections in ruminants.

### Compositions

The compositions of the present invention can be formulated following accepted convention to include acceptable carriers for animals, including humans, such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

The compositions of the present invention can further comprise one or more additional immunomodulatory components such as, e.g., an additional adjuvant or cytokine, among others. Non-limiting examples of such additional adjuvants that can be used in the vaccine of the present invention include the RIBI adjuvant system (Ribi Inc., Hamilton, Mont.), Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, and Avridine lipid-amine adjuvant. Other immunomodulatory agents that can be included in the vaccine include, e.g., one or more interleukins, interferons, or other known cytokines.

The routes of administration for the adjuvant compositions include parenteral, oral, oronasal, intranasal, intratracheal, topical, subcutaneous, intramuscular, transcutaneous, intradermal, intraperitoneal, intraocular, intravenous, and lingual administration. Any suitable device may be used to administer the compositions, including syringes, droppers, needleless injection devices, patches, and the like. The route and device selected for use will depend on the composition of the adjuvant, the antigen, and the subject, and are well known to the skilled artisan.

In view of high infectivity of FMD, measures which need to be taken to contain and/or eliminate FMD outbreak are controlled by regulatory authorities, such as, for example, national Ministries of Agriculture and sanctioned by international organizations such as the OIE (International Office of Epizootics). The measures which need to be undertaken in connection with the outbreak may include, without limitations, standstill of animal movements, effective controls on the movement of animal products, including milk, meat, hide, etc, stamping-out policy (slaughter of the animals in affected herd, and, where appropriate, those in other herds which have been exposed to infection by direct animal to animal contact, or by indirect contact with the pathogen). Often the animals in the neighboring herds are vaccinated followed by slaughter.

The inventors have surprisingly discovered that certain immunogenic compositions described herein prevent persistence, which is defined as the presence or shedding of FMD for longer than 28 days after the infection. In certain embodiments, such immunogenic compositions comprise an antigen component and an adjuvant component, wherein the adjuvant component comprises (or consists essentially of or consists of) an emulsion containing an oily phase, said oily phase comprising at least 50% v/v of said immunogenic composition, an immunostimulatory oligonucleotide in the amount of 75-200 μg per dose, and a polycationic polymer in the amount of 75-200 mg per dose; and the antigen component comprises a FMD antigen in the amount equivalent to at least 6 μg of FMD virus per dose.

In certain embodiments, antigen may be present in the amount equivalent to 6-20 μg of FMD virus per dose, e.g., 8-20, 10-20, 12-20, 14-20, 16-20, 18-20, 6-10, 6-12, 6-18, 8-12, or 8-10 μg of FMD virus per dose. The amount of the immunostimulatory oligonucleotide may be, for example, 75-100, 75-125, 75-150, 75-150, 100-200, 100-150, 125-200, 125-175 or 125-150 μg per dose. The polycationic polymer may be present in the amount of, for example, 75-100, 75-125, 75-150, 75-150, 100-200, 100-150, 125-200, 125-175 or 125-150 mg per dose.

The invention, therefore, also provides a method of reducing frequency of FMD persistence in a ruminant infected with FMD comprising administering to said ruminant prior to the infection the immunogenic compositions which comprise an antigen component and an adjuvant component, wherein the adjuvant component comprises (or consists essentially of or consists of) an emulsion containing an oily phase, said oily phase comprising at least 50% v/v of said immunogenic composition, an immunostimulatory oligonucleotide in the amount of 75-200 μg per dose, and a polycationic polymer in the amount of 75-200 mg per dose; and the antigen component comprises a FMD (Foot-and-Mouth Disease) antigen in the amount equivalent to at least 6 μg of FMD virus per dose.

In different embodiments, the amount of the antigen may be equivalent to 6-20 μg of FMD virus per dose, e.g., 8-20, 10-20, 12-20, 14-20, 16-20, 18-20, 6-10, 6-12, 6-18, 8-12, or 8-10 μg of FMD virus per dose. The amount of the immunostimulatory oligonucleotide may be, for example, 75-100, 75-125, 75-150, 75-150, 100-200, 100-150, 125-200, 125-175 or 125-150 μg per dose. The polycationic polymer may be present in the amount of, for example, 75-100, 75-125, 75-150, 75-150, 100-200, 100-150, 125-200, 125-175 or 125-150 mg per dose.

Administration of these immunogenic compositions to ruminants (e.g., cattle, sheep, camels, etc.) allows for the change in herd management practices. In certain embodiments, the vaccinated members of the herd are not slaughtered after a suspected contact with FMD virus.

In alternative (or additional) embodiments, the vaccinated animals are kept in quarantine for a shorter time. Thus, in certain embodiments, the animals suspected of coming in contact with FMD may be kept in quarantine for less than 30 days, e.g., 28 days, or 29 days.

Further, designation of an area as a containment zone means severe limitations of prohibition on movement of animals or animal products from the containment zone, generally, 30 days or more. Thus, in certain embodiments, the animals suspected of coming in contact with FMD may be moved from the containment zone within less than 30 days, e.g., 28 days or 29 days from the suspected contact with FMD.

In the embodiments where the antigen component entails a genetically engineered FMD antigen, e.g., as described above, it is possible to differentiate vaccinated from infected animals. Therefore, in additional embodiments, the herd management methods (or method of reducing frequency of FMD persistence in a ruminant infected with FMD).

In other words, the immunogenic compositions, in certain embodiments comprising an antigen component and an adjuvant component, wherein the adjuvant component comprises (or consists essentially of or consists of) an emulsion containing an oily phase, said oily phase comprising at least 50% v/v of said immunogenic composition, an immunostimulatory oligonucleotide in the amount of 75-200 μg per dose, and a polycationic polymer in the amount of 75-200 mg per dose; and the antigen component comprises a FMD antigen in the amount equivalent to at least 6 μg of FMD virus per dose may be used for herd management wherein, upon suspected contact with FMD infection, the vaccinated members of said herd are not slaughtered; and/or quarantined for 0-30 days after the suspected contact and/or moved beyond the infected premises within 30 days of the suspected contact.

In different embodiments, the amount of the antigen may be equivalent to 6-20 μg of FMD virus per dose, e.g., 8-20, 10-20, 12-20, 14-20, 16-20, 18-20, 6-10, 6-12, 6-18, 8-12, or 8-10 μg of FMD virus per dose. The amount of the immunostimulatory oligonucleotide may be, for example, 75-100, 75-125, 75-150, 75-150, 100-200, 100-150, 125-200, 125-175 or 125-150 μg per dose. The polycationic polymer may be present in the amount of, for example, 75-100, 75-125, 75-150, 75-150, 100-200, 100-150, 125-200, 125-175 or 125-150 mg per dose.

The invention will be further described in the following non-limiting examples.

### EXAMPLES

### Example 1. Preparation of Antigens

Two methods were used to prepare the antigens: Hollow Fiber Filtration and PEG precipitation.

PEG (poly-ethylene glycol) precipitation methods have been known in the art. Briefly, BHK-21 cells were infected with the FMD virus. Then (24-36 h later) the cells were lysed by freeze-thawing, and cell lysate was clarified of cell debris by low speed centrifugation (500×g). PEG was added (8% w/v) to the supernatant containing both structural and non-structural proteins. The mixture was incubated for 12-18 hr at 4° C. During this incubation, FMDV particles associate with the PEG. Antigen was recovered by centrifugation at 16,000×g and collection of the precipate pellet containing PEG and virus. The supernatant, containing cellular and viral non-structural proteins was discarded. The pellet, to which the virus particles are bound, was then washed with small volumes of buffer to elute the FMDV particles from the PEG.

An additional method described herein is based on hollow-fiber concentration, of FMDV culture supernatants. The steps of this method consist of successive filtration arrangement to remove first the cell debris and large material from the cultures (BHK-21 cells infected with the FMD virus and lysed by freeze-thawing). The culture material was pumped successively through a 10 μm capsule filter, a 4.5 μm capsule filter, then finally through a 0.8 μm/0.2 μm filter. This filtrate was then concentrated using a hollow fiber ultrafiltration cartridge that allows particles smaller than 0.01 μm to flow through the membrane. FMDV particles and many non-structural proteins remain in the column circuit while liquid and smaller proteins go through the membrane into the waste. The column circuit was run until the concentrate reaches the desired volume, normally a ten-fold concentration.

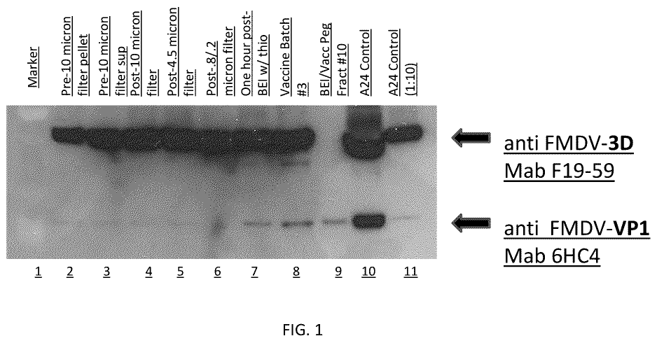

FIG. 1 is a Western blot illustrating the difference in quality between the PEG precipitated and hollow fiber concentrated antigens. Hollow fiber concentrated antigen contains large amounts of structural and non structural proteins as illustrated in this FIGURE by western blot staining using an antibody specific for protein 3D, the largest FMDV non-structural proteins and antibody specific to capsid protein (structural protein). In contrast, PEG-precipitated antigens (lane 9) contained structural protein but did not contain detectable levels of 3D protein.

### Example 2. Effects of FMD Vaccines Adjuvanted with TXO

### Animals and Sample Collection

Six- to eight-month-old Holstein steers weighing 180-230 kg were used in this study. The animals were free of FMDV-reactive antibodies as determined by 3D ELISA test prior to vaccination as determined later from serum samples taken on Day 0. All 28 animals were commingled in one room in a BSL-3-Ag animal testing facility. The animals were fed complete ration pellets or alfalfa cubes, with water and salt blocks available ad libitum. Animals were acclimatized five days to the facilities prior to Day 0. Animals were previously treated with Bovi-Shield GOLD® 5, Micotil® 300, Liquamycin® LA-200® and Dectomax®. Groups of animals (n=4 each) with consecutive ear tag numbers were assigned to a treatment group.

No adverse events were documented following vaccination.

Serum separator blood tubes to obtain serum samples were collected at Days 0 (before vaccination), 4, 7, 14, 21 (before challenge), 24, 28, 31 and 42 from all animals. The serum samples were kept frozen until tested for the presence of neutralizing antibodies against FMDV in a serum neutralization assay (reported as the reciprocal of the last serum dilution to neutralize 100 TCIDs of homologous FMDV in 50% of the wells) or to study the anti-3Dpol response (by means of a competitive Enzyme-Linked Immunosorbent Assay).

As recommended by the OIE (“Manual of Diagnostic Tests and Vaccines for Terrestrial Animals”), challenge of vaccinated cattle for vaccine efficacy was by needle inoculation by the intradermal lingual (IDL) route. At 21 days post-vaccination, all vaccinated and naïve animals were inoculated IDL with 10,000 BTID₅₀ (50% bovine tongue infectious doses) of homologous FMDV A₂₄ Cruzeiro divided as 4 inoculations of 0.1 ml/each with 2,500 BTID₅₀/0.1 ml. All animals were followed for 10 days post-challenge to assess development of clinical disease as expressed by fever, nasal secretion, salivation, loss of appetite and/or lameness. Clinical evaluation for the presence of hoof vesicles was performed with sedation (xylazine given IM at 0.22 mg/kg so as to maintain sternal recumbency for the duration of the procedure) at day 21 (before inoculation) and days 24, 28 and 31. The sedative was reversed with tolazoline, IV, at a dose of 2 mg/kg.

### Vaccines

Antigens were prepared as described in Example 1. Antigen stock solutions contained 5.51 μg/ml antigen prepared by hollow fiber filtertration (Prep A) or 10.26 μg/ml antigen prepared by PEG precipitation (Prep B).

The details of the immunogenic compositions administered to the animals are provided in Table 1. Each group contained four animals.

TABLE 1
Study Design

| | | | | Volume injected, |
|---|---|---|---|---|
| Group | Antigen | Amount/5 ml | Adjuvant/5 ml | ml, IM |
| T01 | None | N/A | PBS (Neg control) | 5 |
| T02 | FMDV (Prep | 8 μg | Light Mineral oil- | 5 |
| | B)-PEG ppt. | | SPAN ® 80 | |
| T03 | FMDV (Prep | 2 μg | TWEEN ® 80 | 1.25 |
| | B) PEG ppt. | | DEAE Dextran | |
| | | | (100 mg); | |
| T04 | FMDV (Prep | 0.5 μg | SEQ ID NO: 8; | 0.3125 |
| | B)-PEG ppt. | | 75% pure: 100 μg | |
| T05 | FMDV (Prep | 8 μg | | 5 |
| | A)-Hollow | | | |
| | fiber filt.- | | | |
| T06 | FMDV (Prep | 2 μg | | 1.25 |
| | A)-Hollow | | | |
| | fiber filt. | | | |
| T07 | FMDV (Prep | 0.5 μg | | 0.3125 |
| | A)-Hollow | | | |
| | fiber filt. | | | |

The immunogenic compositions of groups T02 through T06 were homogenized on the day of vaccination and administered to the animals on Day 0.

Persistence was measured as the presence or absence of virus (either FMDV viral RNA and/or infectious FMDV) determined using both viral isolation and quantitative rRT-PCR. The primers used for the quantitative rRT-PCR were as follows:

Forward (SEQ ID NO: 28):
GACAAAGGTTTTGTTCTTGGTCA
Reverse (SEQ ID NO: 29):
TGCGAGTCCTGCCACGGA
Taqman probe: (FAM reporter, TAMRA quencher,
SEQ ID NO: 30)
TCCTTTGCACGCCGTGGGAC

Serum neutralizing titers to FMDV are summarized in Table 2.

TABLE 2
Serum Neutralizing Titers

| | Serum Neutralizing Titer | | |
|---|---|---|---|
| Treatment | Day 0 | Day 21 | Day 42 |
| T01 | 0.45 ^{a} | 0.45 ^{a} | 2.62 ^{ab} |
| T02 | 0.45 ^{a} | 1.64 ^{c} | 2.84 ^{b} |
| T03 | 0.45 ^{a} | 0.90 ^{b} | 2.39 ^{ab} |
| T04 | 0.45 ^{a} | 0.76 ^{b} | 2.74 ^{ab} |
| T05 | 0.45 ^{a} | 1.55 ^{c} | 2.28 ^{a} |
| T06 | 0.45 ^{a} | 0.81 ^{b} | 2.36 ^{ab} |
| T07 | 0.45 ^{a} | 0.54 ^{a} | 2.68 ^{ab} |

^{a,b,c} Treatment groups with same letter within each day are not significantly different at alpha = 0.05

Signs of FMDV were scored as presence (1) or absence (0) of hoof vesicles, i.e., a presence of a vesicle on a single hoof produced the score of 1, the presence of vesicles on only 2 hooves produced score of 2 and vesicles on all 4 hooves produced a score of 4. Once an animal received a score of 4, it was considered to have a score of 4 for the duration of the study.

The scores from individual animals for each hoof and for each day of examination are shown in Table 3. In Table 4, a summary of each animal's scores according to whether any hoof was positive is presented.

TABLE 3
FMDV Vesicle Scoring individual Animal Listing

| | | Day of Study | | | | | | | |
|---|---|---|---|---|---|---|---|---|---|
| | | 21 | | | | 24 | | | |
| | | Location | | | | Location | | | |
| | | LEFT | LEFT | RIGHT | RIGHT | LEFT | LEFT | RIGHT | RIGHT |
| Treatment | Animal | FORE | REAR | FORE | REAR | FORE | REAR | FORE | REAR |
| T01 | R14- | 0 | 0 | 0 | 0 | 0 | 1 | 0 | 1 |
| | 84 | | | | | | | | |
| | R14- | 0 | 0 | 0 | 0 | 1 | 1 | 1 | 1 |
| | 85 | | | | | | | | |
| | R14- | 0 | 0 | 0 | 0 | 0 | 1 | 0 | 0 |
| | 86 | | | | | | | | |
| | R14- | 0 | 0 | 0 | 0 | 1 | 1 | 1 | 1 |
| | 87 | | | | | | | | |
| T02 | R14- | 0 | 0 | 0 | 0 | 0 | 0 | 0 | 0 |
| | 72 | | | | | | | | |
| | R14- | 0 | 0 | 0 | 0 | 0 | 0 | 0 | 0 |
| | 73 | | | | | | | | |
| | R14- | 0 | 0 | 0 | 0 | 0 | 0 | 0 | 0 |
| | 74 | | | | | | | | |
| | R14- | 0 | 0 | 0 | 0 | 0 | 0 | 0 | 0 |
| | 75 | | | | | | | | |
| T03 | R14- | 0 | 0 | 0 | 0 | 0 | 0 | 0 | 0 |
| | 76 | | | | | | | | |
| | R14- | 0 | 0 | 0 | 0 | 0 | 1 | 0 | 0 |
| | 77 | | | | | | | | |
| | R14- | 0 | 0 | 0 | 0 | 0 | 0 | 0 | 0 |
| | 78 | | | | | | | | |
| | R14- | 0 | 0 | 0 | 0 | 0 | 0 | 0 | 0 |
| | 79 | | | | | | | | |
| T04 | R14- | 0 | 0 | 0 | 0 | 0 | 0 | 0 | 0 |
| | 80 | | | | | | | | |
| | R14- | 0 | 0 | 0 | 0 | 0 | 0 | 0 | 0 |
| | 81 | | | | | | | | |
| | R14- | 0 | 0 | 0 | 0 | 0 | 0 | 0 | 0 |
| | 82 | | | | | | | | |
| | R14- | 0 | 0 | 0 | 0 | 0 | 0 | 0 | 0 |
| | 83 | | | | | | | | |
| T05 | R14- | 0 | 0 | 0 | 0 | 0 | 0 | 0 | 0 |
| | 60 | | | | | | | | |
| | R14- | 0 | 0 | 0 | 0 | 0 | 0 | 0 | 0 |
| | 61 | | | | | | | | |
| | R14- | 0 | 0 | 0 | 0 | 0 | 0 | 0 | 0 |
| | 62 | | | | | | | | |
| | R14- | 0 | 0 | 0 | 0 | 0 | 0 | 0 | 0 |
| | 63 | | | | | | | | |
| T06 | R14- | 0 | 0 | 0 | 0 | 0 | 0 | 0 | 0 |
| | 64 | | | | | | | | |
| | R14- | 0 | 0 | 0 | 0 | 0 | 0 | 0 | 0 |
| | 65 | | | | | | | | |
| | R14- | 0 | 0 | 0 | 0 | 0 | 0 | 0 | 0 |
| | 66 | | | | | | | | |
| | R14- | 0 | 0 | 0 | 0 | 0 | 0 | 0 | 0 |
| | 67 | | | | | | | | |
| T07 | R14- | 0 | 0 | 0 | 0 | 0 | 0 | 0 | 0 |
| | 68 | | | | | | | | |
| | R14- | 0 | 0 | 0 | 0 | 0 | 0 | 0 | 0 |
| | 69 | | | | | | | | |
| | R14- | 0 | 0 | 0 | 0 | 0 | 0 | 0 | 0 |
| | 70 | | | | | | | | |
| | R14- | 0 | 0 | 0 | 0 | 0 | 0 | 0 | 0 |
| | 71 | | | | | | | | |
| | | Day of Study | | | | | | | |
| | | 28 | | | | 31 | | | |
| | | Location | | | | Location | | | |
| | | LEFT | LEFT | RIGHT | RIGHT | LEFT | LEFT | RIGHT | RIGHT |
| Treatment | Animal | FORE | REAR | FORE | REAR | FORE | REAR | FORE | REAR |
| T01 | R14- | 1 | 1 | 1 | 1 | 1* | 1* | 1* | 1* |
| | 84 | | | | | | | | |
| | R14- | 1* | 1* | 1* | 1* | 1* | 1* | 1* | 1* |
| | 85 | | | | | | | | |
| | R14- | 1 | 1 | 1 | 1 | 1* | 1* | 1* | 1* |
| | 86 | | | | | | | | |
| | R14- | 1* | 1* | 1* | 1* | 1* | 1* | 1* | 1* |
| | 87 | | | | | | | | |
| T02 | R14- | 0 | 0 | 0 | 0 | 0 | 0 | 0 | 0 |
| | 72 | | | | | | | | |
| | R14- | 0 | 0 | 0 | 0 | 0 | 0 | 0 | 0 |
| | 73 | | | | | | | | |
| | R14- | 0 | 0 | 0 | 0 | 0 | 0 | 0 | 0 |
| | 74 | | | | | | | | |
| | R14- | 0 | 0 | 0 | 0 | 0 | 0 | 0 | 0 |
| | 75 | | | | | | | | |
| T03 | R14- | 0 | 0 | 0 | 0 | 0 | 0 | 0 | 0 |
| | 76 | | | | | | | | |
| | R14- | 0 | 1 | 0 | 0 | 0 | 1 | 0 | 0 |
| | 77 | | | | | | | | |
| | R14- | 0 | 0 | 0 | 0 | 0 | 0 | 0 | 0 |
| | 78 | | | | | | | | |
| | R14- | 0 | 0 | 0 | 0 | 0 | 0 | 0 | 0 |
| | 79 | | | | | | | | |
| T04 | R14- | 0 | 0 | 0 | 0 | 0 | 0 | 0 | 0 |
| | 80 | | | | | | | | |
| | R14- | 0 | 0 | 0 | 0 | 0 | 0 | 0 | 0 |
| | 81 | | | | | | | | |
| | R14- | 0 | 0 | 0 | 0 | 0 | 0 | 0 | 0 |
| | 82 | | | | | | | | |
| | R14- | 0 | 0 | 0 | 0 | 0 | 0 | 0 | 0 |
| | 83 | | | | | | | | |
| T05 | R14- | 0 | 0 | 0 | 0 | 0 | 0 | 0 | 0 |
| | 60 | | | | | | | | |
| | R14- | 0 | 0 | 0 | 0 | 0 | 0 | 0 | 0 |
| | 61 | | | | | | | | |
| | R14- | 0 | 0 | 0 | 0 | 0 | 0 | 0 | 0 |
| | 62 | | | | | | | | |
| | R14- | 0 | 0 | 0 | 0 | 0 | 0 | 0 | 0 |
| | 63 | | | | | | | | |
| T06 | R14- | 0 | 0 | 0 | 0 | 0 | 0 | 0 | 0 |
| | 64 | | | | | | | | |
| | R14- | 0 | 0 | 0 | 0 | 0 | 0 | 0 | 0 |
| | 65 | | | | | | | | |
| | R14- | 0 | 0 | 0 | 0 | 0 | 0 | 0 | 0 |
| | 66 | | | | | | | | |
| | R14- | 0 | 0 | 0 | 0 | 0 | 0 | 0 | 0 |
| | 67 | | | | | | | | |
| T07 | R14- | 0 | 0 | 0 | 0 | 0 | 0 | 0 | 0 |
| | 68 | | | | | | | | |
| | R14- | 0 | 0 | 0 | 0 | 0 | 0 | 0 | 0 |
| | 69 | | | | | | | | |
| | R14- | 0 | 0 | 0 | 0 | 0 | 0 | 0 | 0 |
| | 70 | | | | | | | | |
| | R14- | 0 | 0 | 0 | 0 | 0 | 0 | 0 | 0 |
| | 71 | | | | | | | | |

*Automatically scored as a ‘1’ since all hooves for this animal previously had vesicles on all four hooves.

TABLE 4
FMDV Vesicle Scoring-Any Hoof Location Positive

| | | Day of Study | | | |
|---|---|---|---|---|---|
| Treatment | Animal | 21 | 24 | 28 | 31 |
| T01 | R14-84 | No | Yes | Yes | Yes* |
| | R14-85 | No | Yes | Yes* | Yes* |
| | R14-86 | No | Yes | Yes | Yes* |
| | R14-87 | No | Yes | Yes* | Yes* |
| T02 | R14-72 | No | No | No | No |
| | R14-73 | No | No | No | No |
| | R14-74 | No | No | No | No |
| | R14-75 | No | No | No | No |
| T03 | R14-76 | No | No | No | No |
| | R14-77 | No | Yes | Yes | Yes |
| | R14-78 | No | No | No | No |
| | R14-79 | No | No | No | No |
| T04 | R14-80 | No | No | No | No |
| | R14-81 | No | No | No | No |
| | R14-82 | No | No | No | No |
| | R14-83 | No | No | No | No |
| T05 | R14-60 | No | No | No | No |
| | R14-61 | No | No | No | No |
| | R14-62 | No | No | No | No |
| | R14-63 | No | No | No | No |
| T06 | R14-64 | No | No | No | No |
| | R14-65 | No | No | No | No |
| | R14-66 | No | No | No | No |
| | R14-67 | No | No | No | No |
| T07 | R14-68 | No | No | No | No |
| | R14-69 | No | No | No | No |
| | R14-70 | No | No | No | No |
| | R14-71 | No | No | No | No |

*Automatically scored as Yes since all hooves for this animal previously had vesicles on all four hooves

All animals in T01 (negative control) exhibited hoof vesicles starting on Day 24. On Days 28 and 31, all hooves in all T01 animals were found to have vesicles. In contrast, full protection (i.e., no hoof vesicles) was observed for every group except T03 (2 μg dose of FMDV precipitated with PEG), where one animal (R14-77) received the score of 1 at Days 24, 28, and 31. The effects of the tested immunogenic compositions on persistent infection are illustrated in Tables 5 and 6. Persistence was defined as presence of infectious virus or viral RNA in oesophageal-pharyngeal fluid (obtained using a “Probang” cup) after 28 days post-challenge (day 49 after vaccination, as shown in tables 5 and 6). In Table 5, quantitative rRT-PCR results for individual animals and treatment group back-transformed least square means of FMDV RNA copy numbers per mL from probang samples are shown. In Table 6, results of probang sample virus isolation testing are reported as either positive or negative. The values below 1.87 in table 5 were scored as ‘negative’ due to limit of detection of the assay.

TABLE 5
Probang rRT-PCR Individual Animal Listing and Back-Transformed
Least Squares Means per Treatment Group

| | | Day 38 | Day 42 | Day 49 | Day 52 |
|---|---|---|---|---|---|
| Treatment | | Test | Test | Test | Test |
| Number | Animal | Result | Result | Result | Result |
| T01 | R14-84 | 4.29 | 4.72 | <1.87 | 3.83 |
| T01 | R14-85 | 4.26 | 6.01 | 5.14 | 4.7 |
| T01 | R14-86 | <1.87 | 3.62 | <1.87 | <1.87 |
| T01 | R14-87 | <1.87 | <1.87 | <1.87 | <1.87 |
| Group Mean | | 1.999 | 3.130 | 1.432 | 1.992 |
| T02 | R14-72 | <1.87 | <1.87 | <1.87 | <1.87 |
| T02 | R14-73 | <1.87 | <1.87 | <1.87 | <1.87 |
| T02 | R14-74 | <1.87 | <1.87 | <1.87 | <1.87 |
| T02 | R14-75 | <1.87 | <1.87 | <1.87 | <1.87 |
| Group Mean | | 0.935 | 0.935 | 0.935 | 0.935 |
| T03 | R14-76 | 4.98 | 4.68 | <1.87 | <1.87 |
| T03 | R14-77 | 5.52 | 3.43 | <1.87 | <1.87 |
| T03 | R14-78 | <1.87 | 4.35 | <1.87 | 5.3 |
| T03 | R14-79 | <1.87 | <1.87 | <1.87 | <1.87 |
| Group Mean | | 2.214 | 2.843 | 0.935 | 1.443 |
| T04 | R14-80 | <1.87 | <1.87 | 4.88 | 4.59 |
| T04 | R14-81 | 5.08 | 4.01 | 3.98 | 4.65 |
| T04 | R14-82 | <1.87 | 4.47 | 6.12 | 4.32 |
| T04 | R14-83 | <1.87 | <1.87 | <1.87 | <1.87 |
| Group Mean | | 1.427 | 1.990 | 3.247 | 3.047 |
| T05 | R14-60 | <1.87 | <1.87 | <1.87 | <1.87 |
| T05 | R14-61 | <1.87 | <1.87 | <1.87 | <1.87 |
| T05 | R14-62 | 4.75 | <1.87 | <1.87 | <1.87 |
| T05 | R14-63 | <1.87 | <1.87 | <1.87 | <1.87 |
| Group Mean | | 1.404 | 0.935 | 0.935 | 0.935 |
| T06 | R14-64 | <1.87 | <1.87 | <1.87 | <1.87 |
| T06 | R14-65 | 4.10 | 4.11 | <1.87 | 3.39 |
| T06 | R14-66 | <1.87 | <1.87 | <1.87 | <1.87 |
| T06 | R14-67 | 4.14 | 5.08 | 5.18 | 4.82 |
| Group Mean | | 1.963 | 2.067 | 1.434 | 1.944 |
| T07 | R14-68 | <1.87 | <1.87 | <1.87 | <1.87 |
| T07 | R14-69 | <1.87 | <1.87 | <1.87 | <1.87 |
| T07 | R14-70 | <1.87 | <1.87 | <1.87 | <1.87 |
| T07 | R14-71 | 5.34 | 5.46 | 4.49 | 3.7 |

| | | | | |
|---|---|---|---|---|
| Group Mean | 1.445 | 1.453 | 1.384 | 1.319 |

TABLE 6
Probang Sample Virus Isolation - Individual Animal Listing

| | | Day of Study | | | |
|---|---|---|---|---|---|
| Treatment | Animal | 38 | 42 | 49 | 52 |
| T01 | R14-84 | Pos | Pos | Pos | Pos |
| | R14-85 | Pos | Pos | Pos | Pos |
| | R14-86 | Neg | Neg | Neg | Neg |
| | R14-87 | Neg | Neg | Neg | Neg |
| T02 | R14-72 | Neg | Neg | Neg | Neg |
| | R14-73 | Neg | Neg | Neg | Neg |
| | R14-74 | Neg | Pos | Neg | Neg |
| | R14-75 | Neg | Neg | Neg | Neg |
| T03 | R14-76 | Pos | Pos | Neg | Pos |
| | R14-77 | Pos | Pos | Neg | Neg |
| | R14-78 | Pos | Pos | Pos | Pos |
| | R14-79 | Neg | Neg | Neg | Neg |
| T04 | R14-80 | Pos | Neg | Pos | Pos |
| | R14-81 | Pos | Pos | Pos | Pos |
| | R14-82 | Pos | Pos | Pos | Pos |
| | R14-83 | Pos | Pos | Pos | Pos |
| T05 | R14-60 | Neg | Neg | Neg | Neg |
| | R14-61 | Neg | Neg | Neg | Neg |
| | R14-62 | Neg | Neg | Neg | Neg |
| | R14-63 | Neg | Neg | Neg | Neg |
| T06 | R14-64 | Neg | Neg | Neg | Neg |
| | R14-65 | Pos | Pos | Pos | Pos |
| | R14-66 | Neg | Neg | Neg | Neg |
| | R14-67 | Pos | Pos | Pos | Pos |
| T07 | R14-68 | Neg | Neg | Neg | Neg |
| | R14-69 | Neg | Neg | Neg | Neg |
| | R14-70 | Neg | Neg | Neg | Neg |
| | R14-71 | Pos | Pos | Pos | Pos |

For Group 1 (saline control), three animals were positive at least once for FMDV by rRT-PCR and two animals were always positive for virus isolation.

In Group T02, no animal was ever found to be carrying FMDV by rRT-PCR, but one animal (R14-74) was found to be positive by virus isolation assay at a single time point only (Day 42: day 21 post-challenge) but negative thereafter (Days 49:and 52, indicating the absence of persistent infection. The other animals in T02 did not carry FMDV detectable either by rRT-PCR or by viral isolation assay at day 38 and beyond.

In group T03, one animal (R14-79) was fully protected from FMDV infection, two animals demonstrated the presence of FMDV (either by rRT-PCR or by viral isolation assay) on three or four of the testing days and one animal (R14-77) demonstrated FMDV presence by both tests on Days 38 and 42, but not thereafter.

In group T04, all four animals exhibited persistence of FMDV by one or both tests through Day 52.

In Group T05, one animal (R14-62) demonstrated the presence of the virus only on Day 38 by rRT-PCR but not by virus isolation and and virus was not detected by either test thereafter. FMDV was not detected either by rRT-PCR or by viral isolation assay at any time for the other three animals in group T05.

In Group T06, two animals were fully protected from persistence while the other two were either rRT-PCR or virus isolation positive at every time point examined.

In group T07, three out of four animals were fully protected while one animal (R14-71) was positive for both rRT-PCR and virus isolation at each time point.

Table 7 summarizes the results of persistence experiments. Animals were considered as non-persistent if neitherrRT-PCR or viral isolation assays detected FMDV on both day 49 (28 days post-challenge) and day 52 (31 day post-challenge).

TABLE 7
Frequency of Persistence and Non-Persistence

| Treatment | Persistent % | Not Persistent % |
|---|---|---|
| T01 (saline) | 50 | 50 |
| T02 (FMDV PEG ppt-8 μg) | 0 | 100 |
| T03 (FMDV PEG ppt-2 μg) | 50 | 50 |
| T04 (FMDV PEG ppt-0.5 μg) | 100 | 0 |
| T05 (FMDV Hollow fiber-8 μg) | 0 | 100 |
| T06 (FMDV Hollow fiber-2 μg) | 50 | 50 |
| T07 (FMDV Hollow fiber-0.5 μg) | 25 | 75 |

Only two of the eight animals administered 8 μg of antigen (Groups T02 and T05) ever exhibited the presence of virus and that was for one day only (one each on Days 37 and 42). The other animals in these groups were fully protected Considering that the virus presence was not detected on both 28 and 31 days after infection, none of the animals administered 8 μg of antigen was considered to be persistently infected. Five out of eight animals administered 2 μg of antigen (Groups T03 and T06) exhibited viral persistence. Four out of eight animals eight animals administered 0.5 μg of antigen (Groups T04 and T07) exhibited persistence.

Taken together, these results indicate protection from FMDV viral persistence in animals administered 8 μg antigen, and it also appears that the purification of the antigen by hollow fiber filtration is advantageous compared to PEG precipitation. The main difference between the two antigen formulations is the presence of non-structural proteins in addition to structural ones in the hollow fiber filtration formulation. Thus, without being bound by theory, it appears that the quality of the immune response elicited by vaccines where the antigen contains both structural and non-structural proteins, and particularly protein 3D, are more effective in preventive FMDV persistence, as illustrated in Table 8.

TABLE 8
Effect of antigen preparation method on immune response.

| Treatment | Persistent % | Not Persistent % |
|---|---|---|
| T01 (saline) | 50% (2 out of 4) | 50% (2 out of 4) |
| Prep A (hollow fiber, groups | 25% (3 out of 12) | 75% (9 out of 12) |
| T05-T07 Combined) | | |
| Prep B (PEG precipitation, | 50% (6 out of 12) | 50% (6 out of 12) |
| groups, T02-T04 Combined) | | |

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1-29. (canceled)

30. A method of herd management, comprising administering to each member in said herd an immunogenic composition wherein said immunogenic composition comprises: a) antigen component comprising at least 6 μg of FMD (Foot-and-Mouth Disease) virus per dose; b) an emulsion containing an oil; c) 75-200 μg of a CpG-containing immunostimulatory oligonucleotide per dose; d) 75-200 mg of a polycationic carrier per dose, and wherein further, upon suspected contact with FMD infection, the vaccinated members of said herd are not slaughtered and wherein the CpG containing immunostimulatory oligonucleotide comprises a 5′ TLR activation domain and at least two palindromic regions, one palindromic region being a 5′ palindromic region of at least 6 nucleotides in length and connected to a 3′ palindromic region of at least 8 nucleotides in length either directly or through a spacer.

31. A method of herd management, comprising administering to each member in said herd an immunogenic composition wherein said immunogenic composition comprises: a) antigen component comprising at least 6 μg of FMD (Foot-and-Mouth Disease) virus per dose; b) an emulsion containing an oil; c) 75-200 μg of a CpG-containing immunostimulatory oligonucleotide per dose; d) 75-200 mg of a polycationic carrier per dose, and wherein further, upon suspected contact with FMD infection, the vaccinated members of said herd are quarantined for 0-30 days.

32. A method of herd management, comprising administering to each member in said herd an immunogenic composition wherein said immunogenic composition comprises: a) antigen component comprising at least 6 μg of FMD (Foot-and-Mouth Disease) virus per dose; b) an emulsion containing an oil; c) 75-200 μg of a CpG-containing immunostimulatory oligonucleotide per dose; d) 75-200 mg of a polycationic carrier per dose, and wherein further, upon suspected contact with FMD infection, the vaccinated members of said herd are moved beyond the infected premises.

33. The method according to claim 30, wherein the vaccinated members of the herd are not slaughtered and are quarantined for 0-30 days.

34. The method according to any one of claim 30-33, wherein the CpG containing immunostimulatory oligonucleotide comprises a 5′ TLR activation domain and at least two palindromic regions, one palindromic region being a 5′ palindromic region of at least 6 nucleotides in length and connected to a 3′ palindromic region of at least 8 nucleotides in length either directly or through a spacer.

35. The method according to claim 34, wherein CpG containing immunostimulatory oligonucleotide comprises at least one of:
a) an iodo-modified nucleotide;
b) one or more phosphorothioate bonds.

36. The method according to claim 35, wherein the iodo-modified nucleotide is the 5′-nucleotide of said CpG containing immunostimulatory oligonucleotide.

37. The method according to claim 36, wherein the immunostimulatory oligonucleotide comprises one or more phosphorothioate bonds and an iodo-modified nucleotide.

38. The method according to claim 37, wherein the polycationic polymer is diethylaminoethyl (DEAE) Dextran.

39. The method according to claim 38, wherein the oil is a light mineral oil.

40. The method according to claim 39, wherein oily phase comprises 50.01%-55% v/v of the immunogenic composition.

41. The method of claim 40, wherein said immunogenic composition comprises 6-20 μg of FMD (Foot-and-Mouth Disease) virus per dose.

42. The method of claim 40, wherein said immunogenic composition comprises 6-18 μg of FMD (Foot-and-Mouth Disease) virus per dose.

43. The method of claim 40, wherein said immunogenic composition comprises 8-12 μg of FMD (Foot-and-Mouth Disease) virus per dose.
